(19) United States
(12) Patent Application Publication
(10) Pub. No.: US 2026/0130614 A1
(43) Pub. Date: May 14, 2026

(54) FACE-WEARABLE OCULAR STIMULATION DEVICE

(71) Applicant: EVOLUTION OPTIKS LIMITED, Christ Church (BB)

(72) Inventors: Raul MIHALI, Westport, CT (US); John Thomas JACOBSEN, Westport, CT (US)

(21) Appl. No.: 18/996,930

(22) PCT Filed: Jul. 26, 2023
(86) PCT No.: PCT/IB2023/057552

§ 371 Date: Jan. 17, 2025

Related U.S. Application Data
(60) Provisional application No. 63/392,755, filed on Jul. 27, 2022.
(60) Provisional application No. 63/490,926, filed on Mar. 17, 2023.

(51) Int. Cl. A61B 5/16

(52) U.S. Cl. CPC A61B 5/165; A61B 5/163

(57) ABSTRACT

Described are various embodiments of a system and method for eye tracking with real-time feedback. Also described are various embodiments of a face-wearable device operable to provide ocular stimulation to a user wearing the device.

### FIELD OF THE DISCLOSURE

The present disclosure relates to wearable devices, and, in particular, to a face-wearable ocular stimulation device.

### BACKGROUND

Eye tracking systems have found use in a wide range of applications, including the presentation of visual content from position-sensitive displays, and the monitoring of ocular behaviour during the performance of various activities from both expert and non-expert users for post-mortem training purposes.

For example, U.S. Pat. No. 9,041,787 issued May 26, 2015 and entitled ‘Portable Eye Tracking Device’, and International Patent Application No. 2020/253,949 published Dec. 24, 2020 and entitled ‘Systems and Methods for Determining One or More Parameters of a User's Eye’, disclose portable eye tracking systems that can be worn similarly to glasses. Such systems allow for gaze monitoring data to be analysed by external resources for a variety of applications, including the evaluation of an efficiency with which a task was performed. Such applications rely on at least partial completion of an activity and subsequent training sessions following analysis of gaze tracking data to inform training practices.

In other contexts, digital head-mounted devices have gained popularity for, for instance, facilitating various daily actions conventionally performed using a smartphone or computer. For example, U.S. Pat. No. 10,025,379 issued Jul. 7, 2018 and entitled ‘Eye Tracking Wearable Devices and Methods for Use’ describes a camera system wearable as glasses that enables a user to take a picture of a surrounding scene after recognising that a user has opted to do so using a characteristic gaze fixation point. A photograph may then be taken in response to the wearable device itself recognising that the user has gazed at a desired focal point in the field of view. Such examples, as well as other systems providing confirmation of user intention via external devices (e.g. a graphical user interface on computer monitors, or the like), notably provide rudimentary feedback in response to specific observed user actions arising from a specific intent of the user.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a system and method for eye tracking with real-time feedback that overcome some of the drawbacks of known techniques, or at least, provides a useful alternative thereto. Some aspects of this disclosure provide examples of such systems and methods.

A need also or alternatively exists for a face-wearable ocular stimulation device that overcomes some of the drawbacks of known techniques, or at least, provides a useful alternative thereto. Some aspects of this disclosure provide examples of such devices.

In accordance with one aspect, there is provided a system for providing real-time feedback to a user based on monitored ocular behaviour, the system comprising a device body configured to be worn on the head of the user and comprising a stimulation portion disposed proximate a periphery of a field of view of the user when the device body is worn. In one embodiment, the device body has coupled therewith an optical sensor configured to acquire optical data corresponding to at least a portion of an eye of the user, and an optical stimulus distributed along the stimulation portion and configured to provide the user with a guidance stimulus perceptible by the user in the periphery of the field of view. The system further comprises a control processor configured to transmit the optical data to a digital processing resource, receive from the digital processing resource a digital guidance signal corresponding at least in part to a designated ocular behaviour and to an ocular behaviour parameter computed at least in part based on the optical data, and upon receipt of the digital guidance signal, activate the optical stimulus in accordance with the digital guidance signal to guide the user via the guidance stimulus to perform the designated ocular behaviour.

In one embodiment, the stimulation portion is disposed proximate an upper or a lower periphery of the field of view when the device body is worn by the user.

In one embodiment, the optical stimulus comprises a distributed light source spatially distributed along the stimulation portion.

In one embodiment, the distributed light source is configured to provide a spatially localised optical stimulus in accordance with the digital guidance signal to guide the user to look in a designated direction corresponding to the spatially localised optical stimulus.

In one embodiment, the optical stimulus comprises a light directing means coupled with the device body to direct light to be perceived by the user in accordance with the digital guidance signal.

In one embodiment, the system further comprises a motion sensor to acquire motion-related data representative of motion of the device body, wherein the control processor is further configured to transmit the motion-related data to the digital processing resource to generate the digital guidance signal at least in part in response to the motion-related data.

In one embodiment, the system further comprises a digital application executable by the digital processing resource to receive as input the optical data, compute the ocular behaviour parameter based at least in part on the optical data, digitally determine the digital guidance signal based at least in part on the designated ocular behaviour and the ocular behaviour parameter, and transmit the digital feedback signal to the control processor.

In one embodiment, the system further comprises an environmental sensor in communication with the digital processing resource and configured to acquire environmental data representative of an environmental parameter, wherein the digital guidance signal corresponds at least in part to the environmental parameter.

In one embodiment, the system further comprises a locator beacon providing an external device with a frame of reference corresponding to the position of the device body with respect to the external device.

In one embodiment, the ocular behaviour parameter comprises one or more of an observed gaze direction, a gaze pattern, a user fatigue, a lack of attention, a risk of an injury, or a cognitive function.

In one embodiment, the designated ocular behaviour comprises one or more of a preferred gaze direction, a corrective gaze direction, or a corrective gaze pattern.

In one embodiment, the system further comprises an illumination source coupled to the device body to illuminate the eye of the user.

In one embodiment, the system further comprises a haptic device addressable by the control processor to provide the user with a haptic stimulus in response to the digital guidance signal.

In accordance with another aspect, there is provided a face-wearable device operable to guide an ocular behaviour of a user wearing the device, the device comprising a device body wearable on the user's face, an optical sensor disposed on the device body and operable to optically monitor the ocular behaviour of the user from within a peripheral field of view of the user, and an optical stimulator disposed on the device body and operable to provide a direct line-of-sight spatially-variable optical stimulus from the peripheral field of view and perceptible by the user in response to the ocular behaviour to guide the user toward a designated ocular behaviour.

In one embodiment, the device is a lensless device so to provide for a physically unobstructed foveal field of view to the user.

In one embodiment, the device body unobstructively contours the user's foveal field of view so to operatively dispose the optical stimulator within the peripheral field of view.

In one embodiment, the device body comprises a nose resting portion to rest on a user nose bridge, and respective optical stimulator body portions extending down and outwardly therefrom to circumscribe respective user eyes within the peripheral field of view, wherein the optical stimulator is operatively mounted on the respective optical stimulator body portions.

In one embodiment, the face-wearable device further comprises respective ear-engaging portions extending rearwardly from distal ends of the respective optical stimulator body portions so to engage user ears to facilitate face wearing.

In one embodiment, each of the respective optical stimulator body portions comprise respective arcuate structures defining respective concave upward facing surfaces when the device is worn, and wherein the optical stimulator is operatively disposed along the respective concave upward facing surfaces.

In one embodiment, the optical stimulator comprises respective sets of optical illumination devices disposed along the respective concave upward facing surfaces.

In one embodiment, the respective sets of optical illumination devices are disposed to extend at least partially up the nose-resting portion.

In one embodiment, the optical stimulator comprises respective steerable optical stimulators disposed on the optical stimulator body portions to steer respective optical stimulations therefrom.

In one embodiment, the respective steerable optical stimulators are operatively disposed around an apex of the optical stimulator body portions in line laterally with the user's eyes.

In one embodiment, the optical stimulator body portions each define outwardly protruding portions that protrude outwardly away from the user's face when worn, and wherein the respective steerable optical stimulators are operatively disposed on the outwardly protruding portions.

In one embodiment, the optical stimulator comprises a discretely addressable distributed light source.

In one embodiment, the distributed light source is configured to provide a spatially localised optical stimulus to guide the user to look in a designated direction corresponding to the spatially localised optical stimulus.

In one embodiment, the optical stimulator comprises a light directing means coupled with the device body to direct light to be perceived by the user.

In accordance with another aspect, there is provided a face-wearable device operable to provide ocular stimulation to a user wearing the device, the device comprising: a device body wearable on the user's face; and an optical stimulator laterally disposed across the user's face in direct line-of-sight within the user's lower and/or upper peripheral field of view and operable to provide a direct line-of-sight laterally-variable optical stimulus from said peripheral field of view laterally stimulating a gaze of the user.

In one embodiment, the optical stimulator is disposed within both of the user's lower and upper peripheral field of view.

In one embodiment, the optical stimulator is adjustable so to be selectively disposed within either of the user's lower or upper peripheral field of view.

In one embodiment, the optical stimulator is adjustable so to be selectively disposed within either the user's lower peripheral field of view or both the user's lower and upper peripheral field of view.

In one embodiment, the optical stimulator is operable in accordance with a designated spatially variable optical stimulation sequence corresponding with a designated oculomotor test.

In one embodiment, the oculomotor test comprises a cognitive impairment test.

In one embodiment, the cognitive impairment test comprises at least one of a smooth pursuit, a saccade or an optokinetic nystagmus test.

In one embodiment, the optical stimulator is selectively operable in accordance with any of a set of designated spatially variable optical stimulation sequences corresponding with respective oculomotor tests.

In one embodiment, the face-wearable device further comprises an eye tracker for tracking an oculomotor response of the user to the optical stimulation sequence.

In one embodiment, the eye tracker comprises at least one of a camera, a pupil tracker or a gaze tracker.

In one embodiment, the optical stimulator is operable in accordance with a designated spatially variable optical stimulation sequence corresponding with a designated user attention enhancement protocol.

In one embodiment, the optical stimulator comprises respective light strip portions disposed to at least partially circumscribe said lower and/or upper peripheral field of view of each eye.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIGS. 1A to 1F are schematics illustrating various perspective views of an exemplary wearable device for providing real-time feedback to a user based on monitored ocular behaviour, in accordance with one embodiment, and FIGS. 1G and 1H are computer-generated images of a wearable device having an alternative stimulus configuration from that of FIGS. 1A to 1F, in accordance with one embodiment;

FIG. 2 is a diagram illustrating various components of exemplary systems for providing real-time feedback to a user based on monitored ocular behaviour, in accordance with various embodiments;

FIG. 3 is a diagram illustrated an exemplary method for real-time feedback to a user based on monitored ocular behaviour, in accordance with one embodiment;

FIG. 4 is a schematic illustrating an exemplary application of a wearable device providing real-time feedback to a user based on sensed environmental data, in accordance with one embodiment;

FIG. 5 is a schematic illustrating an exemplary application of a wearable device providing a frame of reference for position-sensitive displays, in accordance with one embodiment;

FIG. 6 is a photograph of exemplary components of an exemplary wearable device for providing a stimulus to a user based on monitored behaviour, in accordance with one embodiment;

FIG. 7 is a table of exemplary applications for a wearable device and exemplary associated parameters that may be assessed therefor, in accordance with various embodiments;

FIGS. 8A and 8B are screenshots of exemplary user interfaces of a digital application associated with a wearable device, in accordance with some embodiments;

FIG. 9 is a perspective view of a face-wearable ocular stimulation device, in accordance with one embodiment;

FIG. 10 is a front elevation view of the face-wearable ocular stimulation device of FIG. 9 in which a swivel mechanism associated with a selective upper-peripheral field of view optical stimulator is shown in operation;

FIG. 11 is a perspective view of the face-wearable ocular stimulation device of FIG. 10 in which the selective upper-peripheral field of view optical stimulator has been disposed for operation;

FIG. 12 is a perspective view of the face-wearable ocular stimulation device of FIG. 9, operated in accordance with a smooth pursuit oculomotor stimulation sequence, in accordance with one embodiment; and

FIG. 13A is a perspective view of the face-wearable ocular stimulation device of FIG. 9 operated in accordance with an optokinetic nystagmus (OKN) assessment sequence, in accordance with one embodiment, whereas FIG. 13B schematically illustrates a visual pattern sequence replicated for this assessment using the device.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

Various systems are known in the art for tracking the eyes or gaze of a user, and, broadly speaking, may be generally grouped into two categories. The first, referred to herein as ‘remote’ eye trackers, track the two-dimensional (2D) or three-dimensional (3D) position of the eyes or pupils of a user relative to the eye tracker or a related system, and are characterised in that they are not directly coupled with or worn on the user's head, residing rather as system external to the user. Such systems may be a useful in, for instance, systems or methods for presenting visual content that is sensitive to the location of the viewer (e.g. when content is intended to be presented specifically at the location of the user's or users' eye(s)).

For example, and without limitation, visual content provided by a light field system is often rendered in accordance with a ray tracing process, wherein ray vectors are computed between the 3D position of the eye or pupil of the user and individual pixels or pixel groups of a digital display screen, in consideration of any intervening optical elements or layers. For such systems, rendered light field content is generally best consumed or perceived within specific spatially-defined regions of space, referred to herein as a ‘view zone’ or ‘viewing zone’. A non-limiting example of such a light field system utilising remote eye tracking is disclosed in Applicant's co-pending U.S. patent application Ser. No. 17/239,385 filed Apr. 23, 2021 and entitled ‘Pupil Tracking System and Method, and Digital Display Device and Digital Image Rendering System and Method using Same’, the entire contents of which are hereby incorporated by reference. In this example, a remote pupil tracker is employed to monitor pupil movement and determine therefrom an operational mode of a light field system by which a preferred view zone position is measured or calculated from eye tracking data. Such aspects may be useful for, for instance, providing 3D content, or light field content that may be perceived in accordance with an alternative perception adjustment. For example, light field content projected within a view zone location determined at least in part using a remote eye tracker may be rendered in accordance with a visual acuity parameter of the user, whereby a visually impaired user may properly perceive content projected within the view zone by the light field display without the aid of prescription lenses.

While remote eye tracking system have the advantage of leaving a user or unencumbered, and may further be applied to simultaneously monitor the position of the eyes of a plurality of users, their accuracy and precision is often limited. For example, the error of a pupil measurement often increases with distance from the tracker sensor (e.g. a camera), resulting in generally reduced performance when a user is farther away. This can present challenges for various eye tracking applications, wherein the ‘remote’ nature of such systems inherently allows for user movement towards or away from the tracker.

The second broad class of eye tracking systems, on the other hand, may be worn, typically on the head or face of the user, and are accordingly referred to herein as ‘head-mounted’ eye or pupil trackers. Such systems may provide increased accuracy and precision of user gaze measurements over remote systems, and further have the advantage that they may be worn continuously as the user performs various activities in different environments. For example, while a remote eye tracker may monitor user eye positions only when the user is physically present near and generally facing the tracker, a head-mounted eye tracker may continuously monitor user gaze patterns throughout the day while the user is relatively stationary, moving between environments, at home, or at work. Further, once worn and calibrated, such trackers are generally in a fixed position relative to the user, information that can be leveraged to, for instance, reduce computational intensity of calculations related to gaze directions, and/or further improve gaze parameter estimation accuracy.

Various commercial head-mounted eye tracking systems have been proposed. For example, U.S. Pat. No. 9,041,787 issued May 26, 2015 and entitled ‘Portable Eye Tracking Device’, and International Patent Application No. 2020/253,949 published Dec. 24, 2020 and entitled ‘Systems and Methods for Determining One or More Parameters of a User's Eye’, disclose portable eye tracking systems that can be worn similarly to glasses. Indeed, such systems are effectively conventional glasses frames with on-board illumination sources and cameras configured to track user gaze. Such systems allow for gaze monitoring data to be analysed by external resources for a variety of applications, including the evaluation of an efficiency with which a task was performed. For example, such systems may be used to compare gaze parameters during the performance of a designated activity by both an experienced professional and an unseasoned worker, and the results of which may be interpreted to improve training of the latter to perform that activity. Naturally, such applications rely on at least partial completion of the activity and subsequent training sessions following analysis of gaze tracking data. In particular, such systems do not enable real-time feedback related to performance based on eye tracking data.

In other contexts, digital head-mounted devices have gained popularity for, for instance, facilitating various daily actions conventionally performed using a smartphone or computer. For example, U.S. Pat. No. 10,025,379 issued Jul. 7, 2018 and entitled ‘Eye Tracking Wearable Devices and Methods for Use’ describes a camera system wearable as glasses that enables a user to take a picture of a surrounding scene after recognising that a user has intentionally opted to do so using a characteristic gaze fixation point. In this example, the user actively ‘selects’ to take a photograph by intentionally gazing a particular ‘Take Photo LED’ on the wearable device until the wearable device changes the colour of the LED to indicate that the photograph feature is active. The photograph is then taken in response to the wearable device itself recognising that the user has intentionally gazed at a desired focal point in the field of view of the on-board and outwards-facing camera. Such examples, as well as other systems providing confirmation of user intentions via external devices (e.g. a graphical user interface on computer monitors, or the like), notably provide rudimentary feedback in response to specific observed user actions conveying intent with respect to specific device functions (e.g. take a picture, confirm gaze-based selection on an icon on a computer monitor). Such systems do not, however, provide, customised and on-device feedback or guidance in real time in response to, for instance, passive or general ocular behaviour observed during performance of various and/or generalised activities.

Various embodiments herein described, on the other hand, provide different examples of head-mounted eye tracking systems or methods that enable the provision of real-time feedback to the user via various stimuli. Generally, various examples herein described relate to the provision of a device body configured to be worn on the head or face of the user, wherein the device body has coupled therewith an optical sensor configured to capture optical data corresponding to at least a portion of an eye of the user. The device body may further be coupled with a stimulation means (e.g. an optical stimulus, audio, haptic feedback, or the like) configured to provide the user with a stimulus in response to a feedback or guidance signal generated at least in part on optical data captured, in real time and during use. It will be appreciated that, in various contexts, ‘use’ of a device may correspond to the wearing of the device during the performance of a specific activity (e.g. driving a car, playing sports, jogging, reading, using a smartphone or computer, cooking, working, or the like), or may more generally refer to any time that the device is worn.

At least in part to this end, various embodiments further relate to the use of a control processor associated with the device body and/or components coupled thereto, wherein the control processor is configured to transmit the captured optical data to a digital processing resource associated with the device body and coupled components (e.g. an additional on-board processor, and/or a processor associated with a remote device such as a smartphone, laptop, or other computing device), and receive in return therefrom a digital feedback or guidance signal corresponding at least in part to an ocular behaviour parameter computed at least in part based on the optical data transmitted. In accordance with some embodiments, such a guidance signal may additionally or alternatively correspond with a designated ocular behaviour, such as a preferred or desired gaze direction. Upon receipt of the digital guidance signal, the control processor may activate the stimulation means (e.g. an optical stimulus) coupled with the device body to provide the user with a corresponding stimulus (e.g. a guidance stimulus) in accordance with the feedback signal.

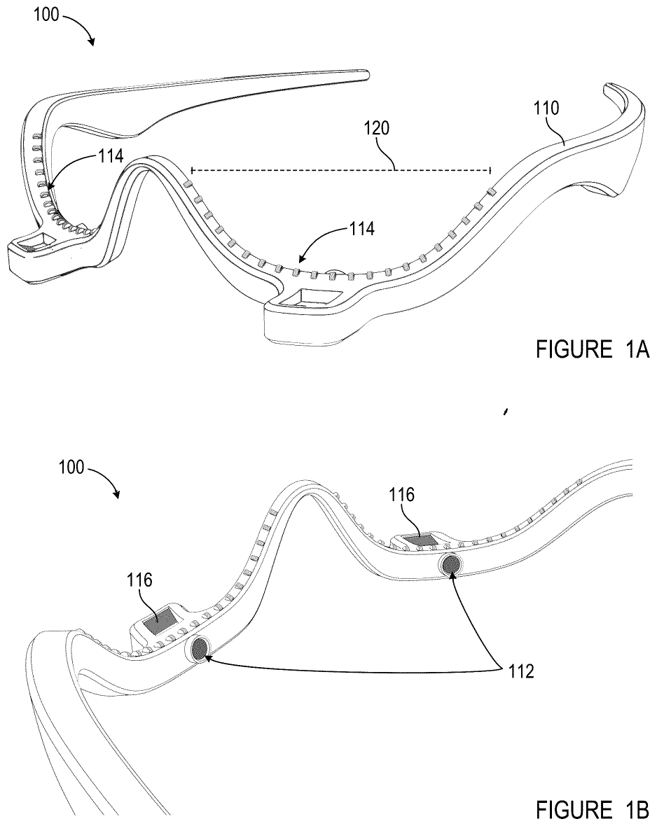

One exemplary embodiment of such a head-mountable system 100 for monitoring user gaze and providing a stimulus to the user in response thereto is presented in FIGS. 1A to 1F. In this example, FIG. 1A is a schematic illustrating a front left perspective view of the system 100 comprising a device body 110 that is configured to be worn on the face of a user similarly to how conventional glasses may be worn. FIG. 1B is a schematic illustrating a rear left perspective view of the front of the system 100 of FIG. 1A. FIGS. 1C to 1F schematically illustrate various alternative views of the system 100 from FIGS. 1A and 1B. It will be appreciated that, as described herein, a device body 110 may comprise various additional or alternative structures or configurations that allow a head-mounted device 100 to be worn while at least a portion of at least one eye of the user is monitored, thereby capturing ocular behaviour, and that allow for the provision of a stimulus that is perceptible to the user during use.

That is, and in accordance with different embodiments, a device body 110 may alternatively relate to or couple with various known wearables, such as a hat, visor, helmet, mask, goggles, glasses, sunglasses, or the like, or may comprise a configuration distinct from known devices or wearable apparel. As various embodiments described herein with respect to the drawings refer to device bodies worn similarly to conventional glasses, for simplicity, a device body 110 may simply be referred to herein as a ‘frame’ 110. However it will be appreciated that various embodiments may relate to alternative device body configurations, and/or that a device body 110 may be reconfigurable or adaptable to be worn as a complement to another form of wearable. For example, a device body 110 may be reconfigurable such that it may be equally worn directly on the face of a user during some activities (e.g. walking, conversing, working, using a computer, viewing a display, or the like), as well as coupled to a helmet, goggles, hat, or the like worn by the user during other activities (e.g. skiing, cycling, or the like).

In some embodiments, and as illustrated in FIGS. 1A to 1F, a device body 110 may comprise a generally linear frame structure (e.g. one that does not comprise apertures, such as those in a frame of sunglasses for supporting lenses). However, in other embodiments, a frame or device body 110 may be shaped similarly to the frame of conventional glasses, and may thus be so worn. Accordingly, it will be appreciated that, as described herein, a device body 110 or frame 110 may further support various optically transparent materials, such as prescription lenses, non-prescription materials (e.g. blue-light filtering materials, polarised materials such as those used in polarised sunglasses, or the like), and/or a non-material (e.g. an absence of lenses or other intervening materials between a user's eyes and the surrounding scene). That is, and in accordance with some embodiments, a frame may not necessarily support materials that are conventionally coupled with, for instance, a frame of conventional glasses, although some embodiments may further comprise such intervening optical layers (whether or not they are prescriptive optical layers) between the eyes of the user and a surrounding environment. Accordingly, some embodiments may relate to a device comprising a ‘lensless’ glasses frame, or a lensless linear frame generally configured as, for instance, the lower portion of glasses frames without the upper portion of frame apertures traditionally supporting lenses, as depicted in the illustrative embodiment of FIGS. 1A and 1B.

In accordance with various embodiments, a head-mountable system 100 further comprises an optical sensor 112 configured to capture optical data corresponding to at least a portion of an eye of the user. In the exemplary embodiment of FIGS. 1A to 1F, the optical sensor comprises respective eye tracking cameras 112 disposed within/on the device body frame 110 to capture images of respective eyes of the user. It will be appreciated that, in accordance with other embodiments, other optical sensors may be employed, or may be coupled with a device body 110 in a different manner and/or location on the device body 110.

For example, camera sensors 112 may be disposed at other location(s) along the frame 110 such that, for instance, a gaze direction may be inferred from ocular data acquired thereby. In accordance with embodiments related to, for instance, more traditional glasses frames, camera sensors 112 may be disposed along an upper region of a device body 110 above where a lens is traditionally placed, or indeed in another suitable location on the frames, or supported by or integrated in/on optical layers (e.g. lenses) in turn supported the by device body 110. Additionally, or alternatively, an optical sensor(s) may protrude or extend from a device body 110 to, for instance, provide an improved field of view of one or more eyes of the user without overly impeding the user's view of a surrounding environment. For example, in order to minimise obstruction of a user's field of view, a device frame 110 may generally be configured such that, when worn, frame portions contour the face well below the eyes of the user. A camera(s) may, in some such embodiments, be coupled with the frame to project outwardly from the face to thereby acquire sufficient ocular data to infer a gaze direction, pupil movement, and/or other eye or pupil parameters, in accordance with various embodiments.

For example, in the exemplary embodiment of FIGS. 1A to 1F, the frame 110 comprises protruding portions 116 protruding outwardly away from the face of the user when in use, while the optical sensors 112 are disposed on the inner side of the frames 110. However, in accordance with another embodiment, the sensors 112 may be disposed on the protrusions 116 so to be disposed farther away from the face of the user, thereby, for some device configurations, increasing the ability of the sensor 112 to capture ocular data. Similarly, embodiments related to a device or system akin to conventional wearables, such as a helmet or mask, may comprise elements protruding from the wearable to provide a sensing geometry by which adequate ocular data may be acquired to determine one or more designated ocular parameters (e.g. gaze directions or patterns thereof).

It will be appreciated that, in accordance with various embodiments, various optical sensors, configurations, and/or operational modes may be employed to acquire ocular data pertaining to the eyes of a user to determine an ocular behaviour parameter. For example, ocular data acquisition may relate to any one or more of various means known in the art of eye tracking, a non-limiting example of which may include cornea position tracking utilising the positions of glints or reflections off the eye of light originating from an illumination source. Accordingly, various embodiments may additionally relate to devices, systems, and methods in which an illumination source(s) (e.g. visible, infrared, ultraviolet, or the like, or a combination thereof) is provided, for instance as coupled to a device body 110 and/or as a component of an eye tracking device that includes the optical sensor 112, and/or is disposed in relation thereto. For example, in the exemplary embodiment of FIGS. 1A to 1F, the protruding regions 116 of the body 110 may comprise illumination sources, while eye tracking cameras 112 are disposed at corresponding regions on the inside of the frame 110. In accordance with other embodiments, such positions may, for instance, be reversed, or other configurations may be provided. It will be appreciated that various embodiments as herein contemplated may comprise other aspects known in the art of eye tracking and/or the extraction of ocular data or parameters using optical sensors, such as a wavelength filter (e.g. a filter that selectively allow infrared light to pass), spectral analysers, one or more of various known data processing techniques, such as differentiation techniques, and/or combinations thereof, as well as other aspects known in the art.

Similarly, some embodiments may relate to the detection of pupil positions from optical images or video of the user's eyes, or another means known in the art for determining ocular behaviour parameters of a user related to, for instance, gaze direction, pupil size, blinking, fatigue, a possible cognitive impairment, or the like. As described above, such processes may be employed in accordance with various known aspects of eye tracking, such as the provision of illumination from an illumination source, or the like. Moreover, it will be appreciated that various embodiments relate to systems that may operate in accordance with various modes. For example, and without limitation, a head-mountable system 100 may operate in accordance with a plurality of illumination modes, wherein, for instance, optical data acquired from an optical sensor may be used to inform an amount of illumination to be provided by an on-board illumination source(s) to compensate for a lack of sufficient ambient light to accurately extract ocular behaviour parameters.

In accordance with various embodiments, the acquisition and/or processing, or the control thereof, of optical data by an optical sensor 112 may be executed at least in part by one or more digital data processors (not shown in FIGS. 1A and 1B) on-board the device body 110. For example, a control processor on-board the frame 110 may execute digital data instructions to activate, deactivate, and otherwise control cameras 112 on the device body 110, as well as, in some embodiments, perform processing of optical data for analysis of ocular behaviour. In some embodiments, such an on-board control processor may directly process and/or analyse ocular data to digitally determine one or more ocular behaviour parameters, such as a pupil size, a pupil position, a gaze direction, a gaze direction pattern, or the like, or may be configured to transmit data to an additional processing resource, which in turn may be disposed on, within, or structurally coupled with the device body 110, or reside remotely from the device body 110.

That is, an on-board control processor my directly process ocular data, or may transmit ocular data to an alternative or additional digital processing resource, in accordance with some embodiments. Such a digital processing resource may comprise on-board computing resource coupled in a wired or wireless manner to the device body 110, or indeed may comprise the control processor itself, and/or may be generally worn by the user as, for example, an attachment to the body 110 or strap or like structure coupled therewith, in accordance with some embodiments. In accordance with some, optionally alternative, embodiments, such a digital processing resource may be remote from the head-mounted device 100, such as a smartphone, a laptop, a personal computer, and/or a like computing resource associated with the system 100. For example, and without limitation, a head-mounted system 100 may be in wireless communication with a smartphone similarly worn or in the presence of the user, which may receive ocular data transmitted by the control processor. The smartphone or similar processing resource may process the ocular data directly, or may in turn communicate the ocular data to another processing resource, for instance to perform more complex or data-intensive computations. For example, in accordance with one non-limiting embodiment, a control processor may transmit ocular data to a smartphone carried by the user and having stored thereon a digital application or like digital data instructions executable by a processor associated with the smartphone to process ocular data to compute, from the ocular data, an ocular behaviour parameter. In accordance with some embodiments, the digital application may further serve as a repository or like medium for storing ocular data and/or ocular behaviour metrics associated therewith or processed therefrom, for instance for further and/or future analysis by the user. In accordance with yet other embodiments, such data, whether ocular data received from the system 100 or metrics or behaviours extracted therefrom, may be communicated with an external resource, for instance to perform further digital computations or analysis, or, in some embodiments, for reference by a professional, such as a medical practitioner analysing the same for a potential condition, improvement of a task, a cognitive impairment, or the like.

Accordingly, various embodiments of a head-mounted system 100 may further comprise a means of wirelessly communicating with an external device. For example, a head-mounted system 100 may comprise digital processing resources, hardware, and/or machine-executable instructions which, upon execution, enable wireless communication, such as, and without limitation, Bluetooth™ or other wireless communication protocols. It will therefore be further appreciated that a head-mounted system 100 may be equipped with a power source (e.g. a rechargeable battery) to power various components of the system 100. For example, and without limitation, a device body, such as the frame 110 of FIGS. 1A and 1B, may comprise a charging interface, such as a USB-or like-based jack or portal to facilitate recharging of a battery or like power source on-board the device 100, as will be appreciated by the skilled artisan.

With continued reference to the system 100 of FIGS. 1A to 1F, a head-mounted system 100 may further comprise a stimulation means 114 (e.g. an optical stimulus 114) configured to provide the user with a stimulus (e.g. a guidance stimulus) in response to an ocular behaviour, in real-time or in near-real time. In accordance with various embodiments, such a stimulus may be provided in response to ocular an ocular behaviour that is observed, computed, and otherwise determined by a digital processor based at least in part on ocular data acquired by the ocular sensor 112, and may, in accordance with some embodiments, correspond with a guidance stimulus to guide the user towards executing a designated ocular behaviour (e.g. to gaze in a designated direction).

Generally, a stimulus may be provided by an optical stimulus 114 in accordance with a digital guidance or feedback signal processed by a control processor associated with the optical stimulus 114. For instance, a control processor associated with the optical stimulus 114 (e.g. a processor in control of the optical stimulus 114, or operable to transmit a control signal to the stimulation means, or the like) may comprise the on-board control processor associated with the optical sensor 112, or, in other embodiments, may comprise a processor distinct from that associated with the optical sensor 112. In either case, a control processor (not shown in FIGS. 1A to 1F) may output a feedback signal (either received or directly computed) corresponding to an observed or computed ocular behaviour. The stimulation means 114 may then, in response to the signal, provide a corresponding stimulus that may be perceived by the user.

In the exemplary embodiment of FIGS. 1A to 1F, the optical stimulus comprises a distributed light source comprising respective arrays of light sources 114 each disposed along a respective stimulus portion 120 of the frame 110, wherein each light source of each array 114 is independently addressable to provide a guidance stimulus characteristic of the digital guidance signal for a respective eye of the user. The guidance stimulus is in turn representative of one or more of a designated ocular behaviour (e.g. a preferred or designated gaze direction), an observed ocular behaviour, and/or environmental parameter. For example, and without limitation, observation of a particular ocular behaviour that is not in agreement with a preferred gaze direction for a given scenario may correspond with the activation of a particular light source of each array 114, or a particular combination or colour of light source(s) of array(s) 114, or a temporal or spatial pattern thereof, thereby providing the user with guiding feedback directly corresponding to an exhibited behaviour. For example, and without limitation, an array of light sources 114 may be spatially distributed along the stimulation portion 120 of the frame 110 such that activation of a particular light source of the array 114 is perceptible within a periphery of the field of view of the wearer of the device, and corresponds to a preferred gaze direction of the user based on a particular application. In accordance with another embodiment, the position within the array of spatially distributed light sources may similarly be understood to have a particular meaning, a non-limiting example of which may include that a particular object of interest in the environment is spatially located relative to the user in correspondence with the position of the activated light source within the array of light sources 114 (i.e. the position of the activated light source may ‘guide’ the eye towards an object of interest).

The exemplary embodiment of FIGS. 1A to 1F comprises a device body 110 in turn comprising a stimulation portion 120 disposed proximate a lower periphery of a field of view of the user when the device body is worn. As such, the optical stimulus 114 may provide a guidance stimulus that is perceptible in the lower periphery of the field of view to guide the user to perform a designated ocular behaviour, such as to gaze in a preferred direction. However, other systems configurations are similarly contemplated, in accordance with other embodiments. For example, for device body configurations comprising traditional glasses, goggle frames, or a helmet having a body portion above the eyes of the user, an optical stimulus may be provided proximate an upper periphery of the field of view. Further, while the optical stimulus of 114 of the system 100 comprises a light source distributed in a lower periphery of the field of view, other embodiments comprise a stimulation portion corresponding with other or a great portion of the field of view. For example, traditional glasses frames generally encircling the field of view may comprise a stimulation portion that similarly encompasses the entire periphery, or portions thereof. For instance, one embodiment relates to the provision of an optical stimulus corresponding to respective light sources or arrays thereof in each of the upper, lower, right, and left periphery of the user's field of view. A guidance signal may then initiate activation of one or more of these distributed light sources to guide the user to look one or more of up, down, right, or left. For example, activation of the right and upper light sources may correspond with a guidance signal instructing the user to look towards the upper-right quadrant of their field of view.

While some such embodiments provide guidance to a designated ocular behaviour based on spatial position in the user's field of view, some embodiments additionally or alternatively provide guidance via a colour of the optical stimulus provided. For example, in the abovementioned embodiment comprising respective optical stimuli in the upper, lower, right, and left peripheries of the user's field of view, additional information may be provided by the colour of the light source activated based on, for instance, the degree to which the user is to exhibit the designated ocular behaviour. For instance, a green light observed from the upper optical stimulus may guide the user to perform a minor upwards adjustment in gaze direction, while a red light in the right stimulus guides to user to a drastic adjustment in gaze direction towards the right. Such aspects may be similarly employed within the context of linearly spatially distributed optical stimuli, such as those of FIGS. 1A to 1F, in accordance with some embodiments. For example, activation of a red light source to the right of the optical stimulus 114 may indicated that the designated ocular behaviour for the user to assume lies outside and to the right of the current field of view observed based on eye tracking data, while a green light may correspond to a preferred gaze direction within the field of view, in accordance with one embodiment.

In accordance with another embodiment, FIGS. 1G and 1H are computer-generated images of a wearable device 140 similar to the head-mounted system 100 of FIGS. 1A to 1F. In this example, however, the wearable device 140 does not comprise on-board illumination source, and sensors 152 acquire ocular data from ambient lighting conditions. Further, the stimulus means 154 of the wearable device 140 is configured differently from the stimulus means 114 of the system 100. While again comprising an array of light sources 154, the array 154 is more narrowly distributed along the device frame 150 as compared to the stimulation means 114 spanning a distance 120, in this case being limited to a stimulation portion of the frame 150 directly below the eyes of the user when in use.

It will be appreciated that various other stimulation means may be provided, in accordance with various embodiments. For example, and as will be further described below, the protrusions 116 of the frame 110 in FIGS. 1A to 1F may, in accordance with some embodiments, support or otherwise relate to a micromirror device 116 or other light directing means to provide a stimulus to the user. As schematically illustrated, a stimulus presented via a micromirror device may be provided on a wearable device 100 in addition to another stimulation means 114, or, in accordance with other embodiments, a micromirror or like light-directing means may define an on-board optical stimulus.

While some such configurations comprising micromirror or like devices are herein contemplated, it will be appreciated that various embodiments may generally relate to a face-wearable device (e.g. device 100) operable to guide an ocular behaviour of a user wearing the device 100. Some such embodiments may generally comprise a device body (e.g. device body 110) wearable on the user's face, as well as an optical sensor (e.g. optical sensor 112) disposed on the device body and operable to optically monitor the ocular behaviour of the user from within a peripheral field of view of the user. A face-wearable device may further comprise an optical stimulator (e.g. stimulus 112) disposed on the device body and operable to provide a direct line-of-sight spatially-variable optical stimulus from the peripheral field of view and perceptible by the user in response to the ocular behaviour to guide the user toward a designated ocular behaviour.

In accordance with some such embodiments, a face-wearable device may comprise a lensless device so to provide for a physically unobstructed foveal field of view to the user. Further, some such devices may unobstructively contour the user's foveal field of view so to operatively dispose the optical stimulator within the peripheral field of view. As shown in the exemplary embodiments of FIGS. 1A to 1H, the body 110 of a face-wearable device 100 may comprise a nose resting portion to rest on a user nose bridge, and respective optical stimulator body portions extending down and outwardly therefrom to circumscribe respective user eyes within the user's peripheral field of view. In some embodiments, each of the respective optical stimulator body portions comprises respective arcuate structures defining respective concave upward facing surfaces when the device is worn, wherein the optical stimulator is operatively disposed along the respective concave upward facing surfaces, The optical stimulator 114, in these non-limiting embodiments, is operatively mounted on the respective optical stimulator body portions. In these embodiments, the exemplary face-wearable device further comprises respective ear-engaging portions extending rearwardly from distal ends of the respective optical stimulator body portions so to engage user ears to facilitate face wearing.

In accordance with some embodiments, the optical stimulator of a face-wearable device may comprise, for instance, respective sets of optical illumination devices disposed along respective concave upward facing surfaces, and may be disposed to extend at least partially up a nose-resting portion of the device. In alternative configurations an optical stimulator may comprise a continuous array or strip of light sources disposed along the device body. In accordance with some such or alternative embodiments, the optical stimulator may comprise respective steerable optical stimulators disposed on optical stimulator body portions to steer respective optical stimulations therefrom. For example, and in accordance with one non-limiting embodiment, respective steerable optical stimulators are operatively disposed around an apex of the optical stimulator body portions in line laterally with the user's eyes. In accordance with some such embodiments, the optical stimulator body portions each define outwardly protruding portions that protrude outwardly away from the user's face when worn, wherein the respective steerable optical stimulators are operatively disposed on the outwardly protruding portions.

In accordance with some embodiments, the optical stimulator of a face-wearable device comprises a discretely addressable distributed light source. For example, in accordance with one embodiment, the distributed light source is configured to provide a spatially localised optical stimulus to guide the user to look in a designated direction corresponding to the spatially localised optical stimulus. In accordance with another embodiment, the optical stimulator comprises a light directing means coupled with the device body to direct light to be perceived by the user.

As will be further described below, various stimuli, or combinations thereof, may correspond to various recognised ocular behaviour parameters and/or designated ocular behaviours. Various embodiments thereby provide an improved richness of information provided as feedback or guidance to the user as compared to systems that have previously been contemplated, such as those related to the confirmation of a specific intended action. For instance, and in accordance with various embodiments, a system or method as herein described may relate to the provision of a stimulus in response to generalised ocular behaviour (e.g. ‘passive’ ocular behaviour, rather than ‘intended’ ocular behaviour corresponding with specific predefined actions). That is, rather than rudimentarily confirming a specific intended action, such as the desire of the user to take a picture through specialised and highly localised activation of a single light-emitting diode upon the recognition of a distinct, specific, intentional, and uniquely identifiable pattern of gaze fixation, various embodiments herein described relate to the provision of one of a plurality of characteristic stimuli (and/or patterns thereof) corresponding to one or more of a range of distinct and/or unique digital feedback or guidance signals computed in response to either an active or passive, intentional or unintentional, or, in some embodiments, autonomic or somatic ocular behaviour, or a pattern thereof.

It will be appreciated that while some embodiments indeed relate to the provision one or more of varied and/or unique sets of stimuli to the user based on corresponding ocular behaviours digitally recognised in real time, various embodiments may additionally or alternatively relate to the provision of comparatively simple stimuli in response to the recognition of particular ocular behaviour. For example, and without limitation, a stimulus may comprise the activation of a single stimulus that is either visually, audibly, or haptically perceived by the user, the nature of which is digitally determined in response to designated characteristic behaviours. These and other embodiments will be described below, with reference to various use cases herein contemplated, in accordance with various embodiments.

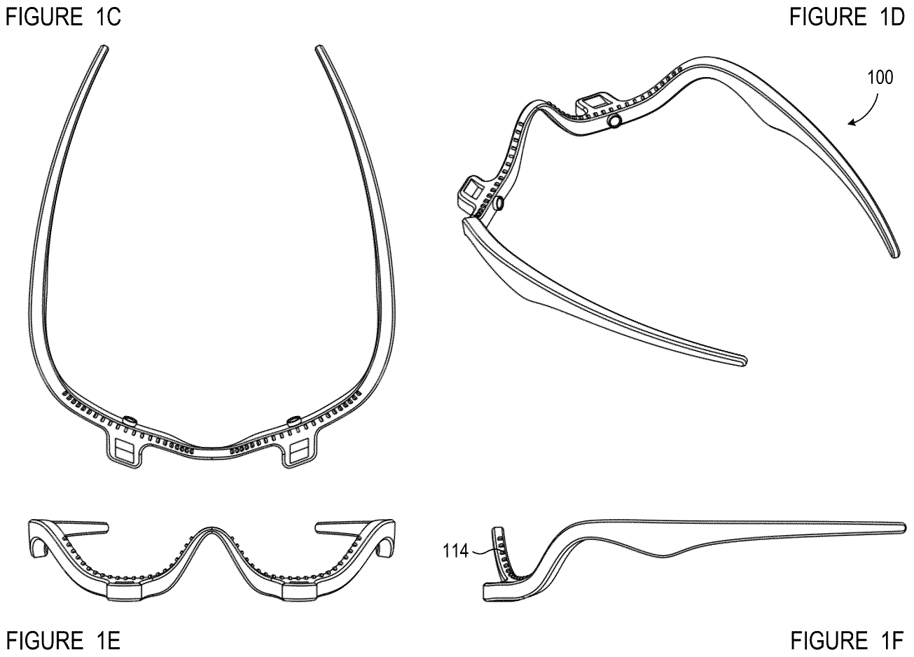

In accordance with various embodiments, FIG. 2 schematically illustrates various aspects of a system 200 for providing feedback in to form of a stimulus provided in real time or near-real time to a user of a head-mounted device 210 in response to observed ocular data. As described above, in this non-limiting example, the device 210 comprises an optical sensor 212 configured to acquire optical data corresponding to at least a portion of an eye of the user. As described above, an optical sensor may comprise a camera generally directed towards the eye of a user to capture the position(s) of glint or reflections off the eye, or another acquisition system known in the art, such as a pupil or eye tracker. The skilled artisan will appreciate that various sensor configurations may be employed, in accordance with various embodiments. For example, a plurality of sensors 212 or cameras 212 may be disposed on a head-mounted frame or device body, wherein one or more of the sensors 212 may address each eye of the user, for example from different monitoring angles with respect to the pupil or cornea of each eye. As described. above, acquisition of optical data by an optical sensor 212 may be facilitated by one or more illumination sources 214, such as an infrared (IR), visible, or ultraviolet (UV) light source disposed on the device 210 so to illuminate the eye in accordance with an optical sensing regime. For example, an illumination source 214 may be activated when ambient light is insufficient to accurately capture ocular data, or a particular wavelength of spectrum thereof may be provided by an illumination source 214 based on the nature of an optical sensor 212 or data to be acquired thereby.

The device 210 further comprises an on-board control processor 216, or a plurality of on-board control processors 216, generally configured to execute digital instructions to control various systems on-board the device 210. In some embodiments, such control processors 216 may be configured to directly process ocular data to assess for various ocular behaviours, and/or may be configured to transmit and receive data related thereto, such as between processors 216 on-board the device 210, or with external processing resources.

For example, a control processor 216 may, via a communication means 218, such as digital data communications hardware and associated software related to BlueTooth technology or an internet-based or like protocol, communicate ocular data and/or parameters related thereto with external resources 220, such as a digital processing resource 222 and/or a digital application 224 associated with an external user device, such as a smartphone app or laptop computer with additional data processing capabilities. It will be appreciated that, in accordance with various embodiments, and depending on, for instance, the computational intensity required or a particular application itself, any or all digital processing may be performed on-board the wearable device 210 via control processor(s) 216 to provide real time feedback to the user in response to observed ocular behaviour. However, for simplicity rather than necessity, various embodiments may be herein described as relating to the use of external processing resources 222 to analyse ocular and/or other forms of acquired data.

It will be appreciated that such processing resources may, depending on, for instance, the particular application at hand, make use of any known or yet to be known processes, networks, hardware, and/or software to perform various computations with respect to acquired data and/or recognise features of interest therein. For example, and without limitation, various embodiments relate to the use of various neural networks, machine learning, or other like processes to extract from ocular or other forms of data various parameters related thereto, for instance to digitally determine a behavioural parameter associated with observed behaviour or external data. Such analysis may result in, for instance, determination of an ocular or other parameter indicative of, for instance, a designated ocular behaviour, cognitive or visual impairment, external stimulus or activity, or the like, that may be indicated to the user via a stimulation means (e.g. an optical stimulus, a characteristic haptic stimulus, or the like) in accordance with a digital feedback or guidance signal generated by one or more of the internal or external processing resources.

Upon recognition of an ocular or other behaviour or parameter related thereto for which a feedback is desired, and/or a designated ocular behaviour (e.g. a desired or preferred user gaze direction), a feedback signal may be generated and executed by a control processor 216 to activate a stimulation means 226. While various stimulation means 226 will be further described below with respect to exemplary applications, non-limiting examples of stimulation means 226 may include a light source, a plurality of light sources, a haptic device, and/or a speaker. It will be appreciated that various embodiments may further relate to a combination of stimulation means 226 to provide stimuli in response to various feedback signals and/or combinations thereof.

As described above, various embodiments of a system 200 for providing feedback to a user in response to observed ocular behaviour using a wearable device 210 relate to an on-board power source 228, which, in accordance with different embodiments, may comprise a rechargeable power source 228 (e.g. a battery rechargeable via a USB or like connection), or a non-rechargeable power source 228, such as a conventional battery. In accordance with some embodiments, a wearable device 210 may additionally or alternatively comprise wireless recharging means, as will be appreciated by the skilled artisan. Further, and in accordance with some embodiments, it will be appreciated that various USB or like connection means, such as those employed for repowering an on-board power source 228, may additionally or alternatively be used as a means of wired communication between the device 210 and an external device, such as a smartphone or other computing device, to enable, for instance, data transfer, device updates (e.g. software or firmware updates), or the like.

While some embodiments relate to the provision of feedback based on ocular data acquired and processed by a wearable device 210 comprising the aspects described above, it will be appreciated that various embodiments may further comprise various additional components to enable additional or alternative features, thereby enabling the device 210 to be used for various alternative or additional applications. For example, and without limitation, a wearable device 210 may optionally comprise a motion sensor 230 to acquire motion-related data related to user or device motion while the device 210 is in use. For instance, various embodiments of a wearable device 210 may comprise an inertial measurement unit (IMU) 230, a gyroscope 230, or like sensor 230 operable to acquire motion-related data, such as user motion or change thereof, user orientation, user position relative to an external frame of reference, or the like. In accordance with some embodiments, such motion-related data may be used in addition or as an alternative to ocular data acquired by an optical sensor 212 to, for instance, provide a feedback to the user in real or near-real time from the stimulus means 226. For example, and without limitation, data related to head motion acquired as a head-mountable device 210 is worn may complement ocular data acquired by an optical sensor 212, and/or an optical behavioural parameter extracted therefrom, to determine a cognitive state of the user, such as if there is a risk that the user is impaired (e.g. from a potential brain injury such as mTBI, inebriation, fatigue, or the like). Upon recognition of a potential risk of impairment through an ocular behaviour parameter extracted from one or more of ocular data or motion-related data, a stimulus may be provided to the user via the stimulation means 226 to accordingly alert the user. In accordance with such and similar embodiments, a wearable device 210 may accordingly be used in applications related to vestibular, ocular, and/or motor screening.

In accordance with some embodiments, a wearable device 210 may additionally or alternatively comprise a locator beacon 232. Generally, a locator beacon may serve to provide a means of locating the wearable device 210 relative to an external device (e.g. an external display 234), such as a display 234 or monitor that, in operation, is sensitive to or otherwise relies upon a knowledge of the position of the wearable device 210 or the user wearing the device. For example, in the context of light field displays which, as described above, may provide visual content that is preferably consumed (and thus projected) in defined regions of space (i.e. spatially defined view zones), accurate knowledge of the location of a user's eye(s) may enhance the experience of viewing the light field content. However, eye trackers associated with the light field display may decrease in accuracy and/or precision with distance from the tracker, resulting in reduced user experience as the display 234 attempts to project light field content in accordance with a view zone location that is determined based on an erroneous user eye or pupil location measurement. Accordingly, various embodiments of a wearable device 210 comprise a location beacon that serves as a ‘frame of reference’ that may be utilised by such systems 234 to improve an estimate of, for instance, the position of a user eye(s) or pupil(s) in 3D space relative to a display.

In accordance with some embodiments, such a locator beacon 232 may serve as a complement to conventional eye tracking device for such display systems, or may replace such tracking systems for various applications. For instance, a locator beacon 232 may serve as a first point of reference to an external display 234, from which user eye position(s) are further refined through the processing of ocular data acquired from an on-board optical sensor 212 tracking, for instance, user pupil locations relative to the known locator beacon position. In accordance with similar embodiments, a locator beacon 232 or like device may similarly extend a range of various tracking devices, for instance by providing a relay point and/or stronger signal than would otherwise be achievable with conventional tracking technologies. However, it will be appreciated that, in accordance with other embodiments, a locator beacon 232 may serve as the sole point of reference for, for instance, a light field display in the determination of a preferred view zone location.

It will be appreciated that, in accordance with various embodiments, a locator beacon 232 may provide a digital signal corresponding to the location of the wearable device to an external device. Accordingly, some embodiments may utilise other device components to establish a device position, such as a position sensor 230 that may additionally serve as a means of acquiring motion-related data. Alternatively, a locator beacon 232 may comprise a distinct digital component from other aspects of the wearable device 210. However, in accordance with other embodiments, a locator beacon 232 may comprise a passive insignia or other like emblem, colour, or feature on a wearable device 210 that may be readily recognised by an image acquisition system associated with an external device 234. For example, and without limitation, an insignia 232 characterised by a colour and/or shape that is readily recognisable to an image recognition system of a light field display 234 may serve as a locator beacon 232, in accordance with some embodiments.

In accordance with some embodiments, a wearable device 210 may additionally or alternatively comprise an on-board environmental sensor 236. For example, and without limitation, an environmental sensor 236 may comprise an outward facing camera 236 disposed on the wearable device 210. Such a sensor 236 may acquire data related to the environment surrounding the user, wherein environmental data may be processed by a control processor 216 and/or an external processing resource 222 to contribute to the determination of a feedback signal to the user of the device 210. For instance, an outward-facing camera 236 may transmit data to an on-board 216 and/or external 222 processor to analyse data in the surrounding environment of a user to inform feedback to the user via a stimulation means 226. For example, and without limitation, an outward-facing camera 236 may acquire video representative of what is seen by the user of a wearable device 236 during performance of a task, such as working, playing a sport, driving, or the like, which is analysed by a processing resource to provide a corresponding stimulus to the user via the wearable device 210 indicative of, for instance, a risk of harm, the location of an object, or the like. In accordance with some embodiments, such an outward-facing camera 236 may further capture data related user behaviour. For example, and without limitation, a camera 236 may acquire image or video data at least in part corresponding to the user's hand(s) while performing a task. Such information may be processed to determine, for instance, if a user is performing the task correctly (e.g. the hand(s) is(are) positioned and/or moved as preferred for an activity). Depending on whether the user is exhibiting the desired or preferred behaviour, a corresponding stimulus may be provided to the user to inform them of, for instance, their accuracy, or to provide a hint or indication of an improved motion, for instance by activating a stimulus in a particular spatial location along the device body. Accordingly, and in accordance with some embodiments, a wearable device 210 may comprise a device for assessing and/or improving motor function for a user.

In accordance with some embodiments, an external environmental sensor 238 may similarly acquire environmental data for processing to provide the user with a corresponding feedback stimulus. For example, and without limitation, a camera 238 external to the wearable device 210 may acquire data representative of the scene around the user, which is in turn digitally processed (e.g. via an external processing resource 222, one associated with a smartphone, or the like), to determine one or more environmental parameters that may warrant indication to the user of the wearable device 210. In accordance with various embodiments, such an external environmental sensor 238 may comprise, for instance, a camera 238 of a smartphone or like device associated with the wearable device 210 (e.g. a smartphone having stored and executed thereon a digital application 224), and/or an alternative or third-party sensor 238 configured and/or disposed to capture events and/or objects in the vicinity of the user. For example, and without limitation, an external environmental sensor 238 may comprise a dashboard camera in a vehicle, or a tracking pod or like device configured with a camera or like sensor programmed to recognise and/or follow objects in the scene, the data related to which a processing resource may analyse to extract information that may be fed back to the user via a stimulation means to, for instance, inform the user as to the location of an object or feature of the scene of interest, or more generally to encourage and/or reinforce mind-body interaction for a particular application or environment. It will be appreciated that such environmental data may, in accordance with different embodiments, relate to data that is independent to ocular data acquired using a wearable device 210, and/or may complement such data in the determination of a digital feedback signal from which a stimulation is provided via a stimulation means 226.

In accordance with various embodiments, a wearable device 210 may additionally or alternatively comprise a means of directing light 240 on-board the device 210. For example, and without limitation, a wearable device 210 may comprise a digital micromirror device configured to direct light towards the user such that it is perceivable. In accordance with some such or alternative embodiments, a light directing means 240 may comprise various additional or alternative optical components, such as lenses, microlenses, microlens array, pinhole arrays, or like optical features that may influence the propagation of light. In accordance with some embodiments, such light directing means may serve as a stimulus to the user, and accordingly man, in accordance with some embodiments, serve as a stimulation means 226 that is activated in response to and in accordance with a digital feedback signal corresponding to an ocular behaviour parameter extracted from ocular data acquired by an optical sensor 212. The skilled artisan will appreciate that such light directing means, such as a digital micromirror device, may be digitally controlled in accordance with various operational parameter to provide the user with various optical stimuli and/or perceptible optical effects, for instance via a control processor 216. Moreover, it will be appreciated that, in accordance with some embodiments, such a light directing means may utilise one or more of ambient light or light from an on-board illumination source 214 to provide, for instance, the user with a feedback stimulus corresponding with any one or more digital feedback signals generated in response to either or both of ocular data or environmental data acquired by an on-board sensor or an external sensor.

With reference again to the external resources 220 of FIG. 2, a system 200 may comprise, avail of, or generally relate to the use of a smartphone or like external device for various aspects or abilities thereof. For example, and without limitation, a smartphone or like device may serve a system 200 as, as described above, a means of processing acquired data (e.g. ocular data, environmental data, motion data, device location data, or the like), and/or as an external sensor and/or display (e.g. a camera 238 acquiring environmental data, a display screen 234 for the display of content, or the like). However, various embodiments may additionally or alternatively relate to the use of a smartphone or like device as a repository for storing, accessing, and/or interacting with data acquired by the system 200 and/or device 210. For example, and in accordance with some embodiments, a smartphone or like device may have stored thereon or otherwise be operable to provide an interface through which the user may access historical data related to the use of a wearable device 210, or indeed to access in real time actively acquired and/or processed data. For instance, various embodiments relate to the acquisition of large amounts of data (e.g. thousands of data points per second), whether related specifically to the user and/or ocular data related thereto, or assessed or extracted behavioural metrics related thereto, which the user may interact with via the external device, interface, or digital application associated therewith.

For example, various embodiments relate to the provision of a digital interface through which a user may interpret and/or analyse data acquired and/or processed from ocular and/or other data during use of the wearable device 210. Such data may be useful in, for instance, providing a means of performing passive neuro-optical training, and/or setting goals and/or tracking progress. It will be appreciated that such user activity may additionally or alternatively relate to customised and/or personalised profiles registered within a digital application and/or with a third-party source. For example, different user-specific profiles may be created and monitored for and/or by each of a plurality of users of a wearable device, and/or as a function of various activities performed therewith. Such a digital platform may further, in some embodiments, enable comparison and/or education related to the performance and/or behaviour of other users, whether such users share the same wearable device 210, or contribute to an online or like platform sharing data and/or performance.

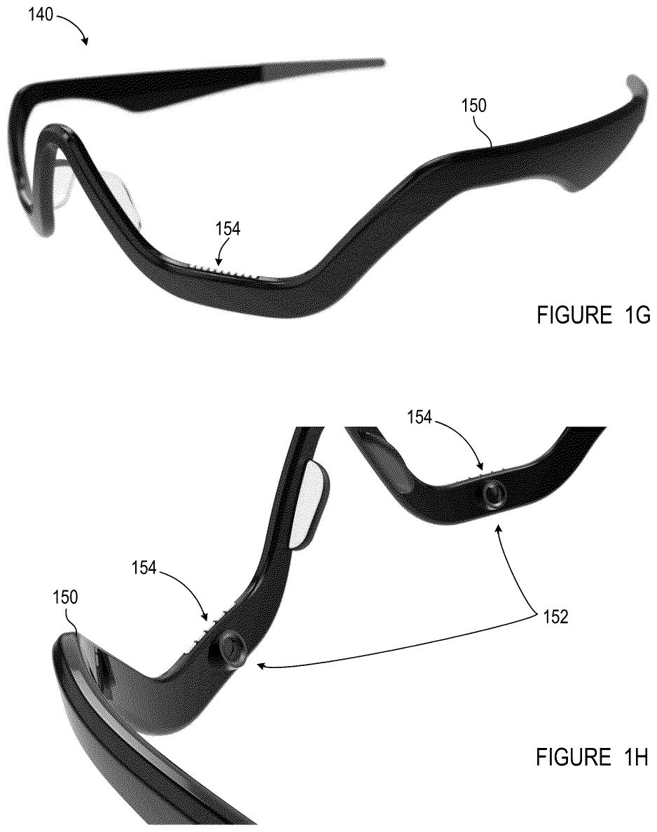

With reference now to FIG. 3, and in accordance with some exemplary embodiments, a process for providing perceptible feedback to a user via a wearable device, generally referred to by the numeral 300, will now be described. It will be appreciated that the schematic diagram of the process 300 is provided for illustrative purposes, only, and that various additional aspects, such as those described above with respect to the system 200 of FIG. 2, may be similarly implemented within the context of the process 300, without departing from the general scope and nature of the disclosure. Moreover, it will be appreciated that various aspects of the process 300, or extensions thereof as herein described, may be similarly implemented as digital process steps executed by one or more digital data processors, and may accordingly relate to corresponding digital data instructions stored on, for instance, a non-transitory computer-readable medium, in accordance with various embodiments.

In the exemplary embodiment of FIG. 3, the process 300 may be executed using a wearable device configured to acquire optical data and provide a stimulus in response thereto, such as the head-mounted device 100 of FIGS. 1A and 1B or the wearable device 210 of the system 200 of FIG. 2. While the process 300 of FIG. 3 may generally be executed by respective components of a wearable device 302 and external processing resources 304, as schematically illustrated, it will be appreciated that various embodiments relate to any or all aspects of the process 300 being executed by a wearable device, depending on, for instance, the particular application at hand.

The process 300 may begin by acquiring optical data 310, for instance via an optical sensor 212. Optical data (and indeed, any other data acquired by a wearable device 210 or external resource 220) may then be transmitted to 312 or otherwise received as input 312 by a digital processor (e.g. a control processor 216 or external processing resource 222) to process 314 the data. The processor may then, at least in part based on the received data, compute a behavioural parameter 316. For example, and without limitation, the processor may, in accordance with various machine learning or other analysis processes, digitally determine a user gaze direction or pattern thereof, a risk of fatigue or impairment, a preferred gaze direction in view of external environmental data, or the like, to determine a corresponding feedback to provide to the user in via a corresponding digital feedback signal 318. The digital feedback signal 318 may then be transmitted 320 or otherwise received as input 320 for a processor and/or stimulation means on board a wearable device to provide a corresponding stimulus 322 to the user of the wearable device 322.

Various examples, applications, and use cases of the processes and systems described above with respect to a wearable device for providing feedback to a user will now be described, in accordance with various non-limiting exemplary and illustrative embodiments. For simplicity, the following examples are described with respect to a wearable device configured similarly to the frame 110 of FIGS. 1A and 1B, although it will be appreciated that various embodiments may relate to alternative device configurations, and may further comprise additional features or components, non-limiting examples of which are described above.

In accordance with one embodiment, a system as herein described may be worn to improve neuro-optic fitness and/or improve the focus of a user performing various activities. For example, a device wearable on the face of the user may comprise inward-facing cameras tracking the positions of both eyes of the user as optical data. Such optical data may be analysed in real time to determine, for instance, gaze direction during performance of various tasks. Such processing may monitor gaze direction over time to determine as an ocular behaviour parameter if a user's attention or focus has waned over the course of an activity. Upon recognising such behaviour, a corresponding feedback signal may be generated to initiate the presentation of an alert stimulus to the user, such as the activation of one or more light sources on disposed on the frame. For example, upon recognition that the user's focus has waned, or a poor posture has been assumed, as recognised by an artificial intelligent or machine learning process, an array of light emitting diodes (LEDs) on the frame may activate in accordance with a designated pattern corresponding to the detected behaviour, thereby alerting the user. By providing such feedback and bringing to the attention to the user indications of an observed behaviour in real time, repeated exposure to such stimuli may ultimately lead to, for instance, improved user focus and/or performance of various activities. Such data may, for instance, be tracked over time, for instance via a smartphone application storing historical data, enabling a user to review performance metrics or compare metrics with those of other users.

In accordance with a similar embodiment, a wearable device may be applied to improve automobile safety. For example, upon recognition of a lack of user focus while driving an automobile, such as if the wearer of the device were to begin to fall asleep or otherwise exhibit distracted or otherwise inattentive behaviour, as determined through analysis of ocular and/or motion data (e.g. gaze patterns, head motion observed via an IMU, or the like) in real time, a stimulation means on the device may be activated to alert the user. For example, a haptic device may be activated to provide the user with a perceptible vibration if it is found that the user's eyes have shut, or if the driver has withdrawn their attention from the road. Similarly a speaker and/or bright light may be activated in response to a feedback signal generated in response to the recognition of a lack of driver focus, in accordance with some embodiments.

Such stimuli may be characteristic of, for instance, the behaviour that was observed via a wearable device. For instance, and without limitation, an alert or like stimulus related to user focus and/or drowsiness while driving, as determined from ocular and/or motion data, may be distinct from a stimulus provided in response to an observed poor posture, or change in posture. For example, observation of a poor posture may result in a sequence or colour of light stimuli, a vibration and/or audio feedback, or the like, provided by a wearable device that is distinguishable from that provided in response to an observed lack of user focus on a task.

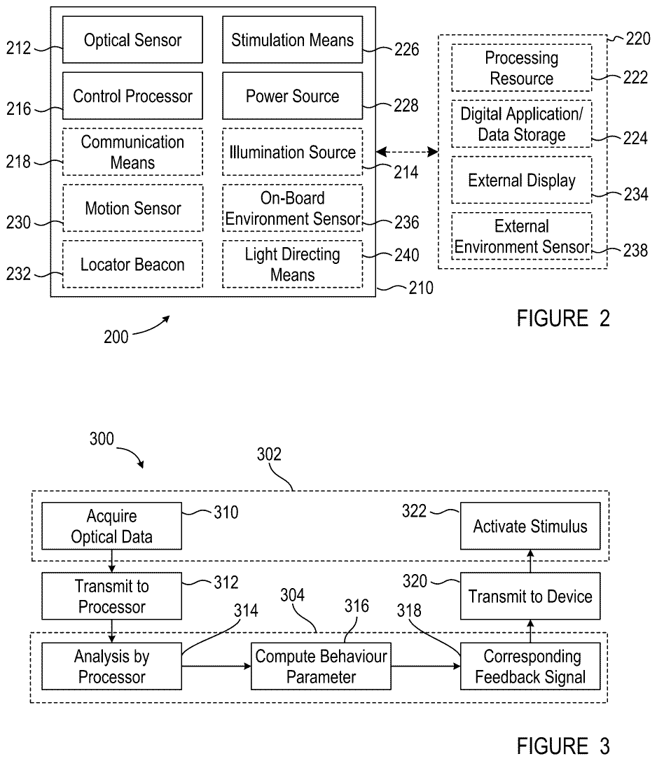

In accordance with another embodiment, a stimulus may be provided to the user of a wearable device as a means of guiding the eye(s) of the user. For instance, FIG. 4 schematically illustrates how a wearable device may provide a stimulus to guide the user in response to environmental data representative of the environment. In this example, a user is wearing a head-mounted device such that their eyes 402 are monitored by eye tracking cameras 404 on-board the device, as described above. However, in this case, an external environmental sensor 406, for instance a camera 406 of a smartphone 408 or tracking pod 408, is also monitoring the scene in front of the user. In accordance with some embodiments, the environmental sensor 406 may additionally or alternatively comprise an outward-facing sensor, such as an outward-facing camera on-board the wearable device. In the exemplary embodiment of FIG. 4, however, the environmental sensor 406 resides externally from the wearable device, and is in wireless communication 410 therewith.

In accordance with one embodiment, the sensor may monitor an environment during performance of a sport or like activity. For example, a tracking pod 408 may be positioned such that it may monitor the position and/or trajectory 414 of a tennis ball 412 during a tennis match. This data may be processed, for example by one or more digital processing resources, trained machine learning models, or the like, to determine a corresponding stimulus to guide the user as to an appropriate response. For instance, in the embodiment schematically represented in FIG. 4, the wearable device comprises an array of LED lights sources 416. Upon recognition that the tennis ball 412 will arrive to the left of the user, an appropriate light source 418 to the left of the array 416 may be activated to guide the user to respond appropriately.

It will be appreciated that, in accordance with various embodiments, any one or combination of light sources may be activated to guide the user. For example, the extent to which a tennis ball 412 will arrive to one side of the user may dictate the position of the light source 418 to activate within the array 416. Further, it will be appreciated that various stimuli may be provided at a given position on the device. For example, a stimulus 418 may be configured to provide various colours of light, the selection of which may correspond with, for instance, how the user has predicted or responded to the environment and/or target. For example, the stimulus 418 may be activated as a red light source at a given position while it is observed that the eyes 402 of the user have not yet appropriately responded to the incoming tennis ball 412. As the user adapts by moving their gaze to the appropriate location, as detected by, for instance, on-board eye trackers 404, the colour of the stimulus 418 may change, for instance to green once the user has appropriately responded.

Accordingly, various embodiments relate to systems and methods for training a user and/or improving their mind and body synergy while performing various tasks. For instance, with respect to the exemplary embodiment of providing a stimulus in response to a tennis ball motion, one may consider that a tennis ball may travel approximately two court lengths per second. Adapting to such speeds requires a high degree of skill and training, which generally requires much time and experience, with limited potential for external feedback to improve. Such feedback is generally limited to post-activity coaching or video review. In accordance with various embodiments herein described, however, such feedback may be provided in real time, or even pre-emptively provided (e.g. in response to a computed trajectory 414) to help guide the user or provide a hint of how to appropriately respond to environmental conditions. Accordingly, such embodiments may improve, accelerate, and/or optimise training for various activities. It will be appreciated that such or similar embodiments may similarly relate to improving training for other activities, non-limiting examples of which may include hockey, football, tennis, golf, jogging, or the like. For example, various embodiments relate to the provision of stimuli in accordance with different operational profiles, such as a designated one or a plurality of profiles corresponding to respective sports or activities. Such profiles may be selected, for instance, prior to performance of the activity via a digital smartphone application associated with the device.

With reference again to FIG. 4, it will be appreciated that an environmental sensor 406 may generally acquire data related to the surrounding environment for processing to digitally determine an appropriate stimulus to provide to the user as guidance, and that such guidance need not necessarily relate to sporting or like activities. For example, and in accordance with some embodiments, the sensor 406 may comprise a dashboard camera configured to detect the presence and/or position of, for instance, pedestrians or other vehicles. Upon recognising a condition warranting the provision of a stimulus to the user, such as if it is detected from eye trackers 404 that the user is not gazing at and/or aware of a potential source of danger, an appropriate stimulus 418 may be provided. As described above, such a stimulus may be provided in a designated location on the device, such as a designated light source 418 of an array 416 to guide the user's gaze to the appropriate area of the scene. In accordance with one embodiment, such stimuli may, for instance, track, mirror, or generally reflect movement of objects of interest in the scene, for instance via sequential activation of different stimuli of an array as, for instance, a pedestrian crosses the street, or the target or object of an activity (e.g. a ball) moves over time, or the like.

In accordance with other embodiments, other everyday activities, such as reading, may similarly benefit from monitoring gaze and providing feedback with respect thereto to improve performance and/or synergy between the mind and body. Moreover, such embodiments may additionally or alternatively improve user experience when, for instance, reading a book in a digital format. For example, a reader wearing a head-mounted device as herein describe may benefit from automatic adjustments of presented content in response to observed ocular behaviour to improve comfort and quality of content, or to identify and correct a potential problem the reader may be developing, such as fatigue and/or a cognitive impairment. Such embodiments may additionally or alternatively relate to the consumption of other visual content, such as that provided by conventional or light field displays. That is, the presentation of content may be adjusted to provide an improved user experience as ocular behaviour is monitored, and any anomalous ocular behaviour may be flagged or otherwise indicated, for instance via an on-board stimulus.

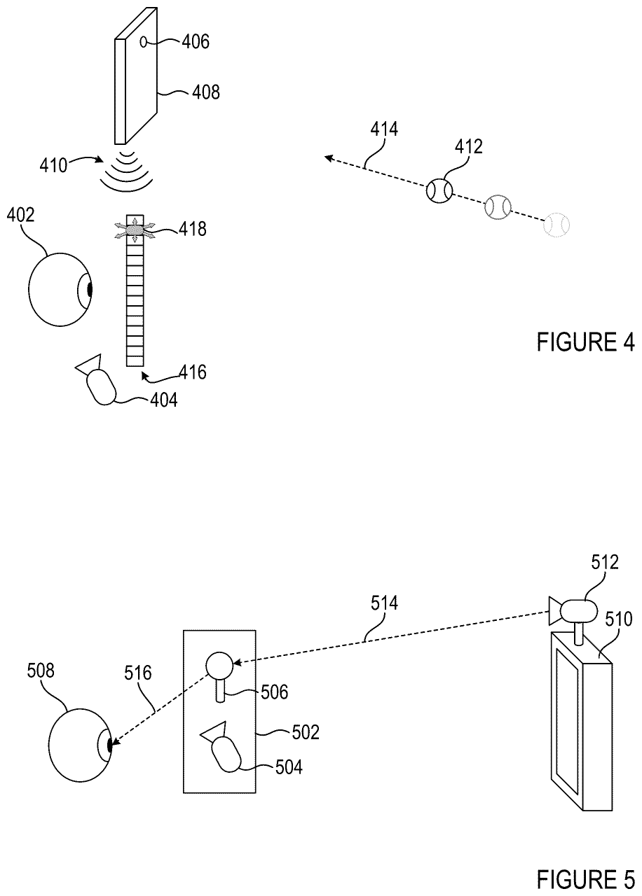

In accordance with yet another embodiment, FIG. 5 schematically illustrates an exemplary system or process for improving estimates of user eye or pupil locations using a wearable device. In this non-limiting example, a wearable device 502 comprises on-board eye tracking functionality 504 and a locator beacon 506 for establishing a frame of reference with respect to the eye(s) 508 and/or pupil(s) of the user. In this illustrated embodiment, the locator beacon 506 serves as a frame of reference relative to a display system 510, such as a light field display, which relies on accurate user eye locations in order to project content thereto. For example, and without limitation, a light field display 510 may be operable to render content in accordance with a perception adjustment corresponding to a visual acuity correction, and/or to be perceived as 3D visual content. In this exemplary embodiment, the light field display 510 has associated therewith a user tracking system 512, such as an eye tracker 512, to determine the position of the user's eye(s) to present content in accordance therewith. As described above, such systems may perform poorly at relatively large distances from the user. However, and in accordance with some embodiments, a locator beacon 506 associated with the wearable device 502 may provide a more accurate frame of reference to determine the position 514 (e.g. the x, y, and z coordinates of the position) of the device 502 relative to the sensor 512 and/or display 510. Upon accurately determining a 3D position 514 of the wearable device 502 relative to the display 510, the 3D position 516 of the eye 508 or pupil relative to the wearable device 502 or beacon 506 associated therewith, whether previously known through a calibration process or directly measured from an eye tracker 504 on-board the device, may be determined to, for instance, accurately project light field content from the display 510 to the eye 508 or pupil. Accordingly, and in accordance with some embodiments, the wearable device 502 may serve to extent a range at which various eye trackers or processes directed to that end may perform accurately and/or precisely.

While the exemplary embodiment schematically illustrated in FIG. 5 comprises a sensor 512 to aid in the determination of the location of the wearable device 502, it will be appreciated that various embodiments do not comprise such a sensor 512. For example, some embodiments relate to the provision of a position of the wearable device 502 directly from a positional sensor or device 506. Such embodiments may thus effectively decouple tracking and positioning of the eye and/or user from a display 510 or like system, removing the need for remote tracking.

Similarly, while the schematic of FIG. 5 illustrates a remote device, such as a light field display 510, various other embodiments herein described may similarly relate to other display systems and/or applications. For example, and without limitation, a wearable device 512 may serve as a frame of reference for eye positions as may be utilised by a cognitive impairment assessment system, such as a portable cognitive impairment assessment system, a dashboard display, a system providing text content for reading, or the like.

In accordance with yet another embodiment, a wearable device may comprise, as a stimulation means or optical stimulus, a light-directing means. For example, with reference again to FIGS. 1A to 1F, protrusions 116 from the frame 110 of the wearable device 100 comprise a digital micromirror device 116 that may direct ambient light and/or light from an illumination source in response to sensed data (e.g. ocular data, environmental data, motion data acquired by an IMU on-board the wearable device 100, or the like). In some embodiments, a stimulus provided by such a light-directing means 116 may further be governed by a microlens array, or other filtering and/or optical layer, such as focusing or colour control elements. For example, one embodiment relates to the provision of perceptible content in the form of light (e.g. ambient or otherwise provided) reflected from a digital micromirror device, optionally onto a light shaping layer, such as a microlens array (MLA) characterised by known and/or calibrated distance and focal parameters, such that light may be projected (e.g. directly or via a light shaping layer) on to the retina of a user as a sharply formed image. Such content may, in some embodiments, comprise light field content provided in accordance with a perception adjustment, such as a visual acuity parameter or optotype, which may be used to, for instance, aid a medical practitioner in the determination of a medical condition, such as a concussion. In accordance with similar embodiments, stimuli provided by such a light directing means may comprise more conventional (e.g. 2D) content. For example, one embodiment relates to the operation of a digital micromirror device in a manner such that rastered 2D visual content is provided through rapid adjustment of mirror elements in response to sensed user and/or environmental data, and/or to guide the user to exhibit a designated ocular behaviour, such as a preferred or designated gaze direction.

It will be appreciated various embodiments herein described may similarly comprise other aspects related to wearable devices, systems, and processes. For example, various aspects of the embodiments herein described may be applied to augmented or virtual reality applications, without departing from the general scope and nature of the disclosure. Further, various aspects of the systems and methods herein described may be similarly applied in the context of other video game platforms and/or e-sports.

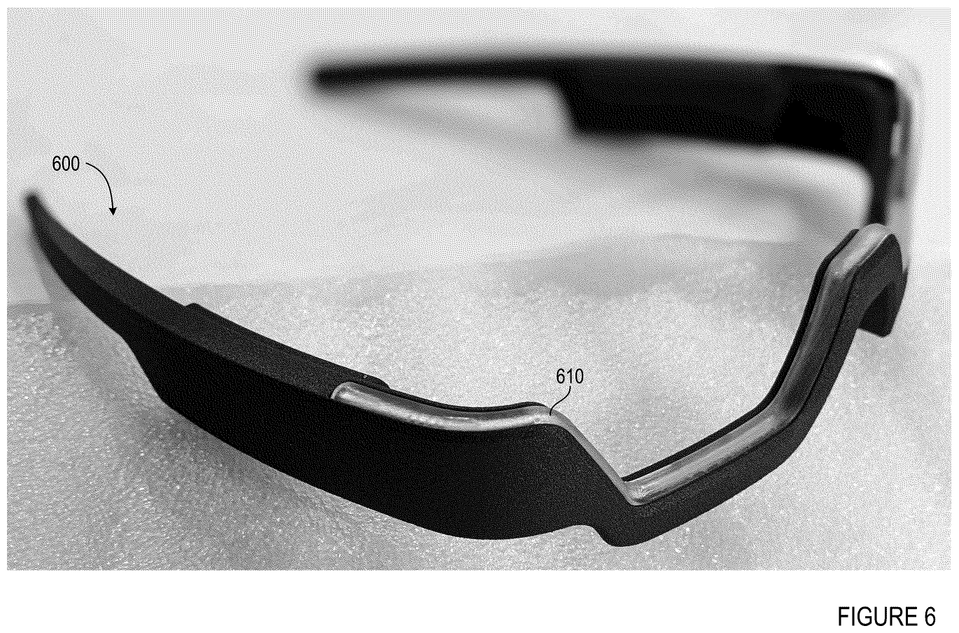

In accordance with yet further embodiments, FIG. 6 is a photograph of an exemplary face-wearable device 600, wherein an optical stimulator thereof is disposed on the device body below a light shaping layer 610. In this example, light from the optical stimulator may be precisely shaped, directed, or otherwise governed as it traverses through the light shaping later 610 to be incident at a precisely designated location, such as the user's retina, or the like. While various embodiments relate to the combination of such a light shaping layer, as noted above, the embodiment of FIG. 6 relates to a device 610 employing a light shaping layer 610 in the absence of a micromirror array.

In accordance with some embodiments, the light shaping layer 610 may comprise, for instance, a microlens array (MLA), a pinhole array, or like device known in the art of, for instance, light field generation, to precisely direct light in accordance with a designated preceptive effect using, for example, a ray tracing process. Accordingly, various embodiments relate to the provision of a face-wearable device comprising an optical source(s) having a designated disposition with respect to, for instance, light shaping elements (e.g. microlenses) of a light shaping layer 610. This may enable, in accordance with various embodiments, the provision of stimuli with a high degree of spatial precision. For example, through the employ of a ray tracing process and compact and/or dense light sources embedded in a face-wearable device with a known disposition relative to an intervening light shaping later 610 or elements thereof, optical stimuli may be provided with a high spatial precision to a designated location (e.g. the user's retina), while minimising or eliminating perceived artefacts, such as undesirable reflections/refractions, halo effects, or the like. Such precision enables the use of such face-wearable devices in, for instance, precision training, concussion and/or autism monitoring and/or therapy, or driving applications, to name a few, in accordance with various embodiments.

In accordance with some such embodiments, optical stimuli, such as LEDs, pixels of miniature displays, or the like, may accordingly be disposed a dense configuration on a device body. For example, in one embodiment, an LED array disposed on the frame may be densely packed so to approximate a linear pixel array, wherein each pixel (i.e. LED) is individually addressable and disposed to enable, for instance, linearly directional control of light emanating from a corresponding light shaping structure, such as a linear MLA structure. Such embodiments may be useful in, for instance, providing linear information (e.g. a suggestion of where a user should gaze in the horizontal direction). It will be appreciated that various embodiments may relate to various configurations of optical stimuli and corresponding light shaping elements.

As a face-wearable device such as the device 600 of FIG. 6 provides a lensless solution when providing visual content (i.e. does not introduce a lens in front of the eye when in use), various embodiments mitigate challenges associated with the vergence-accommodation conflict (VAC) typically experienced with conventional systems (e.g. augmented reality (AR) systems). Such mitigation provides an important advantage over existing virtual/augmented reality systems, particularly for users or classes thereof typically susceptible to discomfort and other issues associated with VAC, such as children.

In accordance with yet further embodiments, solutions proposed herein may additionally or alternatively address perception and/or acuity issues for some users. For example, a person with presbyopia may struggle to perceive content (e.g. read, focus on objects, or the like). One proposed treatment to aid in focusing is the reduction of the pupil size of the effected individual, for instance through the application of eye drops that reduce pupil size. In accordance with some embodiments, such reduction in pupil size to assist in perception of content (e.g. reading) may be facilitated by the devices herein described. For example, one embodiment relates to the provision of a designated stimulus, a non-limiting example of which comprises short and/or bright bursts of light from an optical stimulus and directed to the user's pupil(s) to initiate a rapid reduction in pupil size, thereby improving the user's ability to focus on nearby objects, despite their presbyopia.

In accordance with some such embodiments, such stimuli may be provided as, for instance, a response to observed pupil characteristics or behaviour (e.g. recognition of a lack of pupil constriction, a relatively large pupil diameter as compared to an expected value during performance of a particular task, or the like). Accordingly, and in accordance with at least one aspect, a wearable device configured to provide a stimulus to assist in user acuity may do so dynamically. For example, visual acuity may be dynamically improved for a user by adjusting a frequency and/or intensity of light bursts precisely directed to the eye, as needed, by the wearable device.

It will further be appreciated that various similar embodiments may thus relate to a device for use in optometric phototherapy (i.e. syntonics) applications, wherein a wearable device may provide for the application of selected light frequencies through the eyes. For example, a wearable device may provide one or more selected frequencies of light to the eyes of the user based on a prescription related to the same, in accordance with one embodiment. In accordance with another embodiment, a wearable device may provide such light in response to, for instance, observed gaze dynamics, pupil or eye parameters, and/or other user or ocular behavioural data acquired by the wearable device.

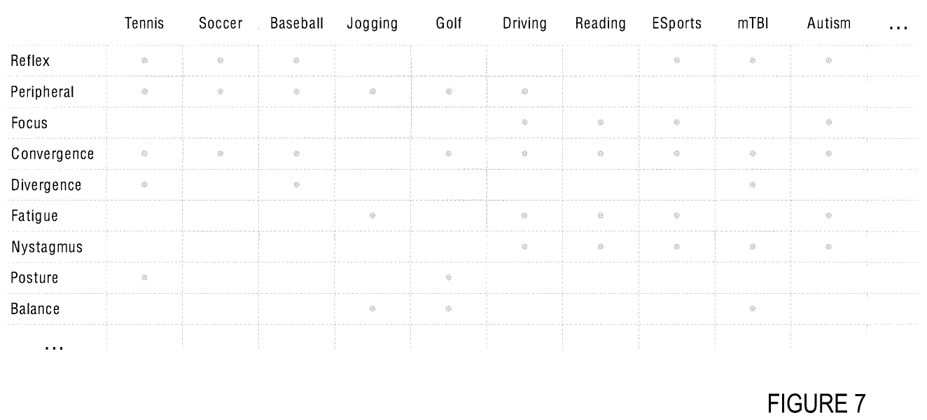

In accordance with various embodiments, and as noted above, a wearable device as herein described may provide support for a wide range of applications, activities, and/or conditions, non-limiting examples of which may include various sports, reading, driving, mTBI, ADHD, red light therapy, and/or autism. Some such applications, as well as additional non-limiting examples, are shown in the table of FIG. 7, wherein non-limiting applications for a wearable device are listed as column headers, and potential non-limiting parameters that may be monitored for each listed application are presented as rows. It will be appreciated that such parameters are listed as corresponding to a given application for exemplary purposes only, and that some such or other applications may monitor and/or assess fewer, additional, or alternative parameters, depending on the particular application at hand.

As the embodiments herein described relate to such diverse applications, various embodiments may additionally or alternatively relate to an ecosystem of digital applications corresponding at least in part to a wearable device as herein described. For example, some embodiments relate to a digital platform (e.g. accessible via a smartphone or networked device) for purchasing and/or accessing digital applications relating to a wearable device as herein described. For example, one embodiment relates to a ‘Neuro-Fitness’ or like digital store for purchasing general device-or application-specific digital programs for use in conjunction with a wearable device. In accordance with some aspects, such a digital environment may relate to the provision of digital applications that are ‘built-in’ or provided with, for instance, purchase and/or use of a wearable device as herein described (e.g. as ‘core’ or general digital applications included with the device). In accordance with additional or alternative embodiments, application-specific, or otherwise-associated applications, may relate ‘premium’ applications that may, for instance, be available for purchase.

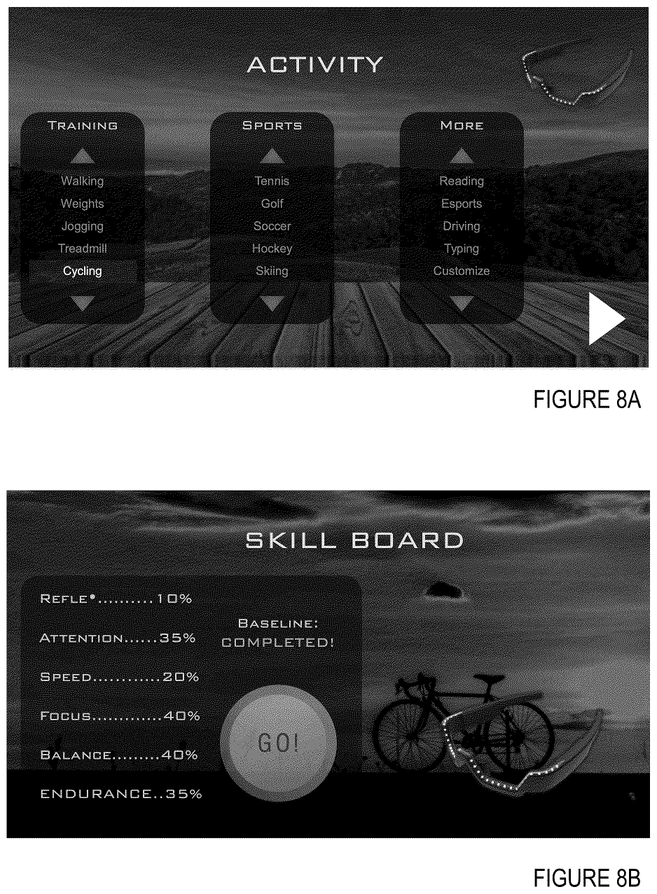

In accordance with one exemplary embodiment, FIG. 8A is a screenshot of an exemplary digital interface where a user may select a digital application based on a use case for which they are using a wearable device. In this example, various non-limiting applications that may be selected by the user are shown in the screenshot. Such digital applications and/or interfaces may be selected from, for instance, previously purchased applications, or may be presented as part of a suite or like ensemble of digital applications provided via, for instance, a smartphone, in association with a wearable device. In the example of FIGS. 8A and 8B, the user has selected cycling as an application. In FIG. 8B, a screenshot of an exemplary display screen shows various scores that a user has achieved as assessed by a wearable device as herein described.

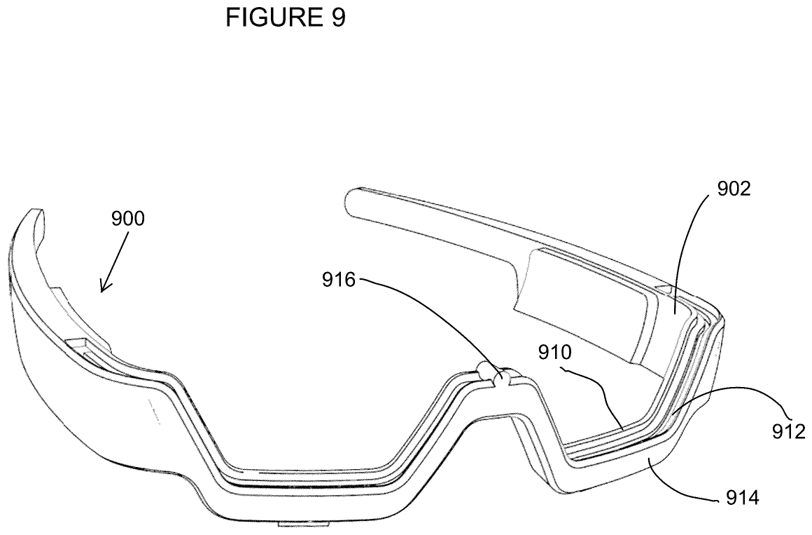

With reference to FIGS. 9 to 11, and in accordance with one embodiment, a face-wearable device, generally referred to using the numeral 900, will now be described in accordance with another embodiment. In this embodiment, the device 900 is again designed to provide an ocular (i.e. visual and/or oculomotor) stimulation to the user wearing the device via an optical stimulator laterally disposed across the user's face in direct line-of-sight within the user's lower and/or upper peripheral field of view. In particular, the device comprises a first set of luminous strips 910 disposed along the frame or body 902 of the device to at least partially circumscribe a lower peripheral field of view of the user for each eye, respectively. Alternatively, the luminous strip may be continuous across the bridge of the nose while being distinctly or discretely driveable on either side thereof to stimulate each eye separately or concurrently. In particular, the luminous strips are disposed so to more or less follow a contour of the user's respective eye regions by respectively descending on each side of the bridge of the nose (where the device body is illustratively configured to rest when in position via appropriately shaped nose bridge-resting device body contour portion and/or bridge-resting pad(s) or the like), extending laterally therefrom below the eye, and then back up again toward the temples.

Generally, the luminous strips 910 comprises a set of discrete lights (e.g. LEDs) or light segments that can be independently, concurrently and/or sequentially activated to produce an ocular stimulation sequence, thus providing a direct line-of-sight laterally-variable optical stimulus from the peripheral field of view of the wearer, for instance laterally guiding, directing and/or stimulating a gaze of the user from this lower peripheral field of view region.

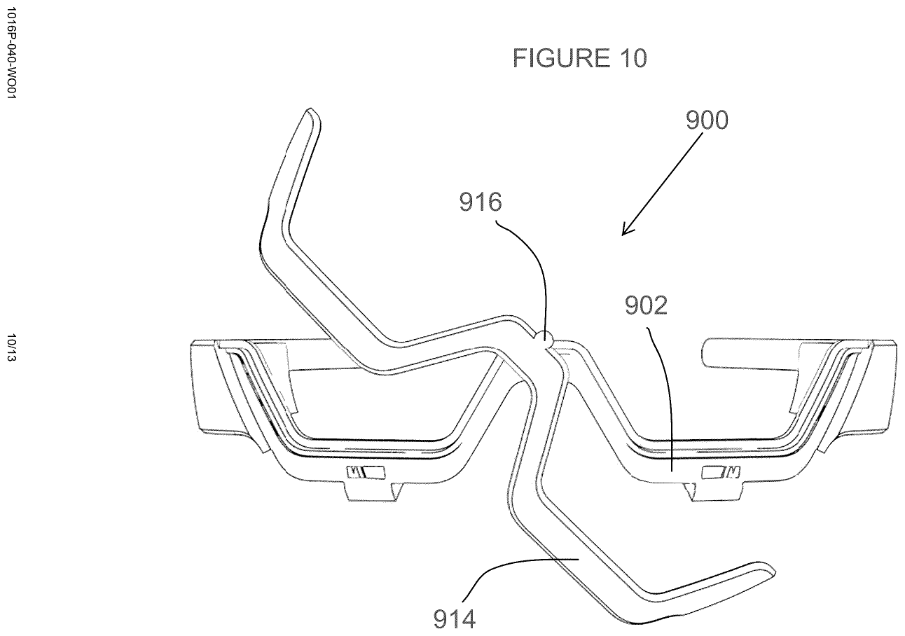

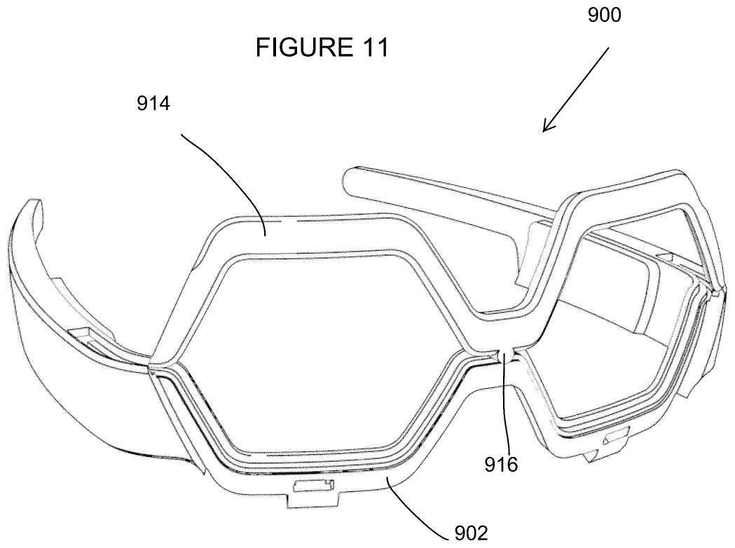

With particular reference to FIG. 10, the device 900 further comprises a second set of luminous strips 912 similarly disposed on a complementary frame or body portion 914 that is mounted to the main frame portion 902 via a swivel mount 916 such that the frame portion 914 can be swiveled from being disposed along a lower peripheral field of view zone (FIG. 9) to being disposed along an upper peripheral field of view zone (FIG. 11). As such, once the swivel frame portion 914 has been swivelled, the device 900 can be used to stimulate the user's eyes from below and/or above. Much as luminous strip 910, luminous strip 912 comprises a set of discrete lights (e.g. LEDs) or light segments that can be independently, concurrently and/or sequentially activated to produce an ocular stimulation sequence, thus providing a direct line-of-sight laterally-variable optical stimulus from the lower (FIG. 9) or upper (FIG. 11) peripheral field of view of the user.

In some embodiments, the device 900 may further comprise one or more eye or pupil tracking cameras and/or illumination devices (e.g. infrared (IR) or near-infrared (NIR) light source and camera) to track an ocular response to the stimulation. Additional stimulation devices, for example so as to produce vibrational, thermal, optical and/or audio stimulation concurrently and/or sequentially with the luminous strip stimulation, may also be provided.

As illustrated, the device 900 can be worn more or less like one would wear typical eyeglass frames, without lenses, so to dispose the eye-framing portions of the device 900, and luminous strips disposed to illuminate more or less vertically therefrom within the lower and/or upper peripheral field of view of the user. As introduced above, the device 900, much as the other embodiments described above, can be used in various applications, for example, to provide different metrics, indicators, and controls for controlling and/or observing oculomotor behaviour. Below, different exemplary oculomotor assessments that may be implemented using the devices described herein, and specifically illustrated within the context of device 900, will now be described. However, it will be appreciated that such assessments are presented for exemplary purposes, only, and that other assessments may similarly be performed, without departing from the general scope or nature of the disclosure. For example, various other oculomotor tests that may be similarly performed are described (in the context of a 2D or 3D display) in Applicant's co-pending International Application No. PCT/US 2022/013564, wherein the 2D oculomotor stimuli provided in those described embodiments can be reconfigured to be provided via the luminous strip(s) 910 (912).

For example, the device 900, in accordance with various embodiments, may be configured and operable to perform saccade assessments, for instance for the purpose of screening for a potential TBI. In one example, a saccade assessment may comprise presenting a stimulus in the form of an illuminated dot (light strip portion or constituent LED(s)) that appears in two different locations. Such an assessment may be automatically performed, for instance via execution of digital instructions stored on the device or accessed thereby, in accordance with preset or designated parameters.

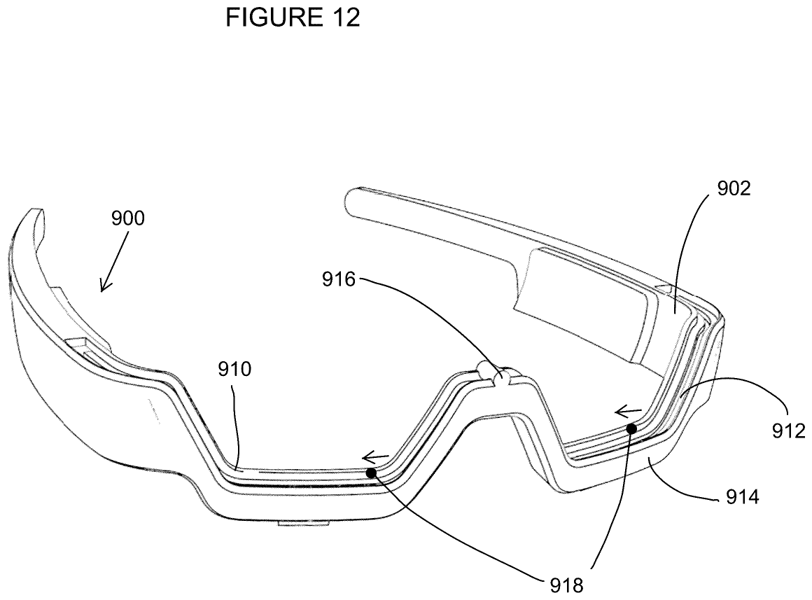

Saccade assessments may be performed in accordance with different modes, which may be selectable via a GUI, or pre-loaded as part of a predetermined battery of tests. In one exemplary assessment, a luminous dot is made to appear at a certain distance from center for a designated amount of time before disappearing, to be relocated at the mirrored position along an axis such that the plane of reference passes through the center. Such a symmetric configuration relates to a predictive saccade test. Using the device 900 of FIG. 12, luminous dots may be presented on either or both the upper and lower luminous strips to provide a level of two-dimensionality to this and other tests.

In accordance with one embodiment, the duration and location of the stimulus are based on a controlled computation of a square wave function derived from a sinusoidal wave function. For example, the desired position and duration of a stimulus presentation may be defined by the practitioner, or predefined in accordance with a designated testing parameter set, to define the amplitude and period of the wave function, respectively. As a saccade test may require only one dot to appear at any given time at a fixed position during the entire duration of its appearance, the sinusoidal wave is replaced with a square wave function, in accordance with various embodiments.

In one embodiment, a saccade assessment may be predictive, wherein the amplitude of a square wave corresponding to stimulus position is constant, and the stimulus alternates between two fixed positions. In accordance with some embodiments, non-predictive saccade tests may be similarly performed. However, as non-predictive saccades relate to the appearance of stimuli in different positions, a random value may be introduced in the computation of the square wave amplitude. For example, the amplitude calculation described above may be multiplied by a random number for each new stimulus position. In one embodiment, the random number is determined from a random number generator, wherein various device parameters are considered in the random number generation.

Another exemplary assessment that may be performed with the devices described herein relates to a smooth pursuit of a stimulus. As with saccade assessments described above, smooth pursuit assessments may involve a luminous stimulation dot or segment that is displaced between two different locations along the luminous strip (e.g. for each eye independently, or for both eyes concurrently). With illustrative reference to FIG. 12, this may comprise, for instance, presenting a luminous dot or segment 918 that moves leftwards to a position specified by a displacement control. Upon reaching the defined destination, the point may then move rightwards (and passing through the centre in some examples) to reach a mirrored or opposite position.

In one embodiment, this motion may be defined by a sinusoidal wave. Accordingly, the particular sequence of continuous positions of the stimulus may be defined by a controlled computation of the sinusoidal wave function. In other words, the position of the dot during such an assessment is defined by the amplitude and period or frequency of the sinusoidal wave function. As with saccade assessments, smooth pursuit may be predictive or not predictive (e.g. the amplitude of displacement changes between cycles).

Various embodiments may further relate to a device operable to perform reaction time assessments. Such assessments may similarly relate to the provision of a stimulus along the luminous strip, wherein, for example, a luminous dot or segment appears for a short time (e.g. for 50 ms). Such assessments may, in accordance with some embodiments, provide a potential biomarker for various conditions, such as a concussion, where concussed users often exhibit an increase in time required to react compared to baseline.

In one embodiment, the reaction time may be computed as the difference in time between a first illumination of the stimulus and the time at which a user performs a reaction, such as clicking a button or otherwise consciously reacting to the stimulus. Time may be recorded as, for instance, the difference in time stamps associated with these events. In accordance with some embodiments, one or more of the presentation time of the stimulus (i.e. how long a dot is presented for) and the delay time between successive presentations of the stimuli may be preset, and may be fixed or variable.

Various embodiments further relate to a device operable to perform optokinetic nystagmus (OKN) assessments. Such assessments of visual function may be particularly useful in, for instance, assessment of children or other users for whom obtaining a reliable response may be challenging. OKN assessments may relate to involuntary eye movement evoked by a repeating pattern stimulus in continuous motion. Such motion may consist of two phases: a smooth phase elicited when the user tracks a target (i.e. slow component velocity or SCV) and saccadic fast movement in the opposite direction (i.e. quick phase or QP), termed as a “resetting event”. This resetting event initiates when the user re-fixates on a newly appearing feature of the stimulus movement. The resulting data output is a sawtooth form when plotting displacement versus time. Various algorithms are known that are aimed at automatically extracting the resulting sawtooth data characteristics of gaze patterns, such as duration, amplitude and velocity estimates.

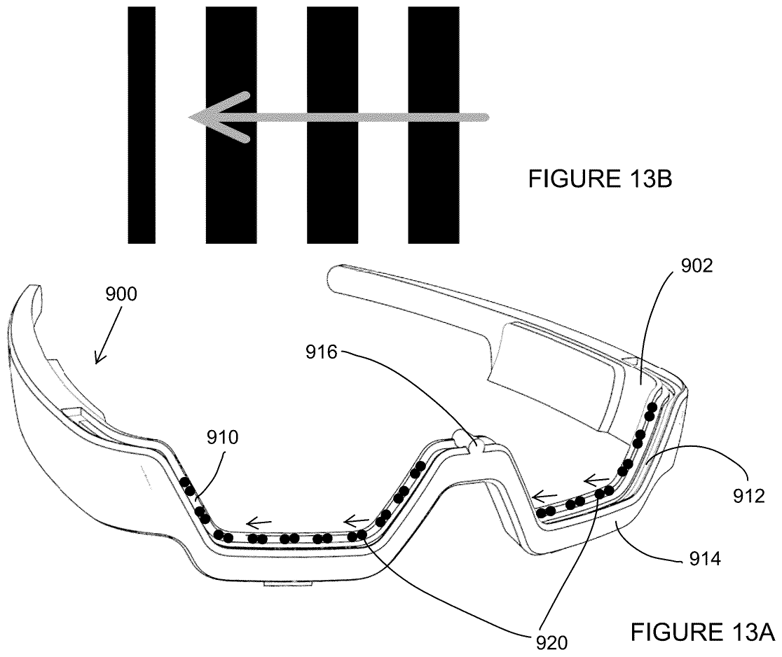

FIGS. 13A and 13B schematically represent OKN assessments presented on device 900, wherein FIG. 13A illustrates a recurring patter of luminous dots 920 scrolling along the luminous strip 910, whereas FIG. 13B illustrates the corresponding luminous pattern more commonly displayed for OKN assessments using a conventional digital display means. As will be appreciated by the skilled artisan, different pattern dimensions (luminous segment lengths), speeds, direction, etc. can be implemented.

During assessment, the user is asked to fix their gaze on an illuminated segment and follow its motion, indicated by arrows in FIG. 13A. Once the gaze reaches the end of the display, the user moves their gaze back to the first illuminated segment of the pattern, and repeats following its motion.

As will be appreciated by the skilled artisan, variations of, and alternatives to the above-noted examples may be considered within the present context to further exand the utility of the herein-proposed embodiments.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A system for providing real-time feedback to a user based on monitored ocular behaviour, the system comprising:
a device body configured to be worn on the head of the user and comprising a stimulation portion disposed proximate a periphery of a field of view of the user when said device body is worn, said device body having coupled therewith:
an optical sensor configured to acquire optical data corresponding to at least a portion of an eye of the user; and
an optical stimulus distributed along said stimulation portion and configured to provide the user with a guidance stimulus perceptible by the user in said periphery of said field of view; and
a control processor configured to:
transmit said optical data to a digital processing resource;
receive from said digital processing resource a digital guidance signal corresponding at least in part to a designated ocular behaviour and to an ocular behaviour parameter computed at least in part based on said optical data; and
upon receipt of said digital guidance signal, activate said optical stimulus in accordance with said digital guidance signal to guide the user via said guidance stimulus to perform said designated ocular behaviour.

2. The system of claim 1, wherein said stimulation portion is disposed proximate an upper or a lower periphery of the field of view when said device body is worn by the user.

3. The system of either one of claim 1 or claim 2, wherein said optical stimulus comprises a distributed light source spatially distributed along said stimulation portion.

4. The system of claim 3, wherein said distributed light source is configured to provide a spatially localised optical stimulus in accordance with said digital guidance signal to guide the user to look in a designated direction corresponding to said spatially localised optical stimulus.

5. The system of any one of claims 1 to 4, wherein said optical stimulus comprises a light directing means coupled with said device body to direct light to be perceived by the user in accordance with said digital guidance signal.

6. The system of any one of claims 1 to 5, further comprising a motion sensor to acquire motion-related data representative of motion of said device body, wherein said control processor is further configured to transmit said motion-related data to said digital processing resource to generate said digital guidance signal at least in part in response to said motion-related data.

7. The system of any one of claims 1 to 6, further comprising a digital application executable by said digital processing resource to:
receive as input said optical data;
compute said ocular behaviour parameter based at least in part on said optical data;
digitally determine said digital guidance signal based at least in part on said designated ocular behaviour and said ocular behaviour parameter; and
transmit said digital feedback signal to said control processor.

8. The system of any one of claims 1 to 7, further comprising an environmental sensor in communication with said digital processing resource and configured to acquire environmental data representative of an environmental parameter, wherein said digital guidance signal corresponds at least in part to said environmental parameter.

9. The system of any one of claims 1 to 8, further comprising a locator beacon providing an external device with a frame of reference corresponding to the position of said device body with respect to said external device.

10. The system of any one of claims 1 to 9, wherein said ocular behaviour parameter comprises one or more of an observed gaze direction, a gaze pattern, a user fatigue, a lack of attention, a risk of an injury, or a cognitive function.

11. The system of any one of claims 1 to 10, wherein said designated ocular behaviour comprises one or more of a preferred gaze direction, a corrective gaze direction, or a corrective gaze pattern.

12. The system of any one of claims 1 to 11, further comprising an illumination source coupled to said device body to illuminate said eye of the user.

13. The system of any one of claims 1 to 12, further comprising a haptic device addressable by said control processor to provide the user with a haptic stimulus in response to said digital guidance signal.

14. A face-wearable device operable to guide an ocular behaviour of a user wearing the device, the device comprising:
a device body wearable on the user's face;
an optical sensor disposed on said device body and operable to optically monitor the ocular behaviour of the user from within a peripheral field of view of the user; and
an optical stimulator disposed on said device body and operable to provide a direct line-of-sight spatially-variable optical stimulus from said peripheral field of view and perceptible by the user in response to the ocular behaviour to guide the user toward a designated ocular behaviour.

15. The face-wearable device of claim 14, wherein the device is a lensless device so to provide for a physically unobstructed foveal field of view to the user.

16. The face-wearable device of claim 14 or claim 15, wherein said device body unobstructively contours the user's foveal field of view so to operatively dispose said optical stimulator within said peripheral field of view.

17. The face-wearable device of any one of claims 14 to 16, wherein said device body comprises a nose resting portion to rest on a user nose bridge, and respective optical stimulator body portions extending down and outwardly therefrom to circumscribe respective user eyes within said peripheral field of view, wherein said optical stimulator is operatively mounted on said respective optical stimulator body portions.

18. The face-wearable device of claim 17, further comprising respective ear-engaging portions extending rearwardly from distal ends of said respective optical stimulator body portions so to engage user ears to facilitate face wearing.

19. The face-wearable device of claim 17 or claim 18, wherein each of said respective optical stimulator body portions comprise respective arcuate structures defining respective concave upward facing surfaces when the device is worn, and wherein said optical stimulator is operatively disposed along said respective concave upward facing surfaces.

20. The face-wearable device of claim 19, wherein said optical stimulator comprises respective sets of optical illumination devices disposed along said respective concave upward facing surfaces.

21. The face-wearable device of claim 20, wherein said respective sets of optical illumination devices are disposed to extend at least partially up said nose-resting portion.

22. The face-wearable device of any one of claims 17 to 21, wherein said optical stimulator comprises respective steerable optical stimulators disposed on said optical stimulator body portions to steer respective optical stimulations therefrom.

23. The face-wearable device of claim 22, wherein said respective steerable optical stimulators are operatively disposed around an apex of said optical stimulator body portions in line laterally with the user's eyes.

24. The face-wearable device of claim 23, wherein said optical stimulator body portions each define outwardly protruding portions that protrude outwardly away from the user's face when worn, and wherein said respective steerable optical stimulators are operatively disposed on said outwardly protruding portions.

25. The face-wearable device of any one of claims 14 to 24, wherein said optical stimulator comprises a discretely addressable distributed light source.

26. The face-wearable device of claim 25, wherein said distributed light source is configured to provide a spatially localised optical stimulus to guide the user to look in a designated direction corresponding to said spatially localised optical stimulus.

27. The face-wearable device of any one of claims 14 to 24, wherein said optical stimulator comprises a light directing means coupled with said device body to direct light to be perceived by the user.

28. The system or face-wearable device of any one of claims 1 to 27, wherein said optical stimulus or stimulator comprises a microlens array for at least partially governing a light field emanated thereby toward an eye or retina of the user.

29. A face-wearable device operable to provide ocular stimulation to a user wearing the device, the device comprising:
a device body wearable on the user's face; and
an optical stimulator laterally disposed across the user's face in direct line-of-sight within the user's lower and/or upper peripheral field of view and operable to provide a direct line-of-sight laterally-variable optical stimulus from said peripheral field of view.

30. The face-wearable device of claim 29, wherein said optical stimulator is disposed within both of the user's lower and upper peripheral field of view.

31. The face-wearable device of claim 29, wherein said optical stimulator is adjustable so to be selectively disposed within either of the user's lower or upper peripheral field of view.

32. The face-wearable device of claim 29, wherein said optical stimulator is adjustable so to be selectively disposed within either the user's lower peripheral field of view or both the user's lower and upper peripheral field of view.

33. The face-wearable device of any one of claims 29 to 32, wherein said optical stimulator is operable in accordance with a designated spatially variable optical stimulation sequence corresponding with a designated oculomotor test.

34. The face-wearable device of claim 33, wherein said oculomotor test comprises a cognitive impairment test.

35. The face-wearable device of claim 34, wherein said cognitive impairment test comprises at least one of a smooth pursuit, a saccade or an optokinetic nystagmus test.

36. The face-wearable device of claim 33, wherein said optical stimulator is selectively operable in accordance with any of a set of designated spatially variable optical stimulation sequences corresponding with respective oculomotor tests.

37. The face-wearable device of any one of claims 33 to 36, further comprising an eye tracker for tracking an oculomotor response of the user to the optical stimulation sequence.

38. The face-wearable device of claim 37, wherein said eye tracker comprises at least one of a camera, a pupil tracker or a gaze tracker.

39. The face-wearable device of any one of claims 29 to 32, wherein said optical stimulator is operable in accordance with a designated spatially variable optical stimulation sequence corresponding with a designated user attention enhancement protocol.

40. Thee face-wearable device of any one of claims 29 to 39, wherein said optical stimulator comprises respective light strip portions disposed to at least partially circumscribe said lower and/or upper peripheral field of view of each eye.
